SEQUENCE LISTING (60 sequences)

SEQ ID NO: 1 - DNA, 9155 bp, Artificial Sequence
  Plasmid BAY02430 encoding a modified Cas9
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcctgcaggcccgggttaattaagcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggaccatggcacccaagaagaagaggaaggtgggcatccacggcgtcccagcagcaatggacaagaagtactccatcggcctcgacatcggcaccaacagcgtcggctgggccgtgatcaccgacgagtacaaggtgccaagcaagaagttcaaggtgctgggcaacaccgaccgtcactccatcaagaagaacctcatcggcgccctcctcttcgactcaggtgagacggccgaggcgacaaggttgaagaggacggcaagaagaaggtacacaagaaggaagaacaggatctgctacctccaggagatcttcagcaacgagatggccaaggtggatgacagcttcttccaccggctagaggagagcttcttggtggaggaggacaagaagcatgagaggcatcccatcttcggcaacatcgtcgacgaggtggcctaccatgagaagtaccccaccatctaccatctccgcaagaagctggtggactcaacggacaaggccgacctccgcctcatctacctcgccttggcccacatgatcaagttcagaggccacttcctcatcgagggcgacctcaaccctgacaacagcgatgtggacaagctcttcatccagctggtgcagacctacaaccagctctttgaggagaaccccatcaacgcctccggcgtcgacgccaaggccatcctctcggcaaggttgagcaagtcaagaaggctggagaacctcatcgctcagctccccggcgagaagaaaaatggcctcttcggcaacctcatcgccttgagcctcggcctcacccccaacttcaagtcaaactttgacctcgccgaggatgccaagctccagctctccaaggacacctacgacgacgacctcgacaacctcctcgcccagatcggcgaccagtacgccgacctcttcctcgccgccaagaacctctcagatgccatcctcctgagcgacatcctccgcgtcaacaccgagatcaccaaggcgccgctctccgcctccatgatcaagaggtatgatgagcaccatcaagatctcaccctcctcaaggccttggtgaggcagcagctacctgagaagtacaaggagatcttcttcgaccagagcaagaatggctacgccggctacatcgacggcggcgcctcccaagaagagttctacaagttcatcaagcccatcttggagaagatggatggcaccgaggagctactggtgaagctcaacagagaagatctcctcaggaagcagaggaccttcgacaacggcagcatccctcaccagatccaccttggtgagctccatgccatcttgagaaggcaagaagacttctaccccttcctcaaggacaacagggagaagatcgagaagatattgacattcaggatcccctactacgtcggacccttggcaagaggcaactcaaggttcgcctggatgacaaggaagagcgaggaaaccatcaccccctggaactttgaggaggtggtggacaagggcgcctcagctcagagcttcatcgagaggatgaccaacttcgacaagaacctccccaacgagaaggtgctccccaagcacagcctcctctacgagtacttcaccgtctacaatgagctcaccaaggtcaagtacgtcaccgagggcatgaggaagccggccttcctctcaggagagcagaagaaggccatcgtcgacctcctcttcaagaccaacaggaaggtcaccgtcaagcagctcaaggaggactacttcaagaagatcgagtgcttcgactctgtggagatctccggcgtggaggacaggttcaacgcctccctcggcacctaccatgatctcctgaagatcatcaaggacaaggacttcctcgacaacgaggagaacgaggacatcttagaagacatcgtcctcaccctcaccctcttcgaggacagggagatgatcgaggagaggctcaagacctatgctcacctctttgatgacaaggtgatgaagcagctcaagagaagaaggtacaccggctggggccgcctctcaaggaagctcatcaacggcatcagggacaagcagagcggcaagaccatcctcgacttcctcaagagcgatggcttcgccaacaggaacttcatgcagctcatccatgatgacagcttgaccttcaaggaggacatccagaaggctcaagtctccggccaaggagattccctccatgagcacatcgccaacctcgccggctcaccggccatcaagaagggcatcctccagaccgtcaaggtggtggatgagctggtgaaggtgatgggccgtcacaagccggagaacatcgtcatcgagatggcaagggagaaccagaccacccagaagggccagaagaactcaagggagaggatgaagaggattgaagaaggcatcaaggagctcggcagccagatcctcaaggagcaccctgtggagaacacccagctacagaacgagaagctctacctctactacctccagaatggccgtgacatgtacgtcgaccaagagctggacatcaacaggttgagcgactacgacgtcgaccacatcgtcccccagagcttcctcaaggatgacagcatcgacaacaaggtgctgacaagatcagacaagaaccgtggcaagagcgacaatgtcccctccgaggaggtggtgaagaagatgaagaactactggaggcagctcctcaacgccaagctcatcacccagaggaagttcgacaacctcaccaaggctgaaagaggaggcctctcagagctggacaaggccggcttcatcaagaggcagctggtggagacaaggcagatcaccaagcatgttgctcagatccttgattcaaggatgaacaccaagtacgacgagaacgacaagctcatcagggaggtcaaggtcatcaccctcaagagcaagctggtgtccgacttcaggaaggacttccagttctacaaggtgagggagatcaacaactaccaccatgctcatgatgcctacctcaacgccgtggtgggcaccgccctcatcaagaagtaccccaagctggagtcagagttcgtctacggcgactacaaggtgtatgatgtgaggaagatgatcgccaagagcgagcaggagatcggcaaggccaccgccaagtacttcttctactcaaacatcatgaacttcttcaagaccgagatcaccctcgccaacggcgagatcaggaagaggccgctcattgagacaaatggtgagactggtgagatcgtctgggacaagggccgagacttcgccaccgtgaggaaggtgctctccatgcctcaagtcaacatcgtcaagaagaccgaggtgcagaccggaggcttctcaaaggagagcatcctccccaagaggaactctgacaagctgatcgcaaggaagaaggactgggaccccaagaagtacggcggctttgattcaccaacggtggcctactccgtgctggtggtggccaaggtggagaagggcaagagcaagaagctcaagagcgtcaaggagctcctcggcatcaccatcatggagagatcatccttcgagaagaaccccatcgacttccttgaggccaagggctacaaggaggtgaagaaggacctcatcatcaagctccccaagtacagcctcttcgagctggagaatggccgcaagaggatgctggcatcagctggagagctccagaagggcaacgagctggccctcccaagcaagtacgtgaacttcctctacctcgcctcccactacgagaagctcaagggctcaccagaagacaatgagcagaagcagctcttcgtggagcagcacaagcactacctcgacgagatcatcgagcagatctccgagttctccaagagggtgatcctcgccgacgccaacctcgacaaggtgctctcagcctacaacaagcaccgtgacaagcccatcagggagcaagctgagaacatcatccacctcttcaccctcaccaacctcggcgccccggcggccttcaagtacttcgacaccaccatcgaccgcaagaggtacacctcaaccaaggaggtgcttgatgccaccttgatccaccagagcatcaccggcctctacgagacaaggatcgacctctcccagctcggcggcgacaagaggccggcggccaccaagaaggccggccaagccaagaagaagaagtgattgctagcacgcgttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcggcgcgccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - protein, 1401 aa, Artificial Sequence [sequence omitted]

SEQ ID NO: 3 - DNA, 9155 bp, Artificial Sequence
  Plasmid BAY02734 encoding Cas9 D10A nickase
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcctgcaggcccgggttaattaagcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggaccatggcacccaagaagaagaggaaggtgggcatccacggcgtcccagcagcaatggacaagaagtactccatcggcctcgccatcggcaccaacagcgtcggctgggccgtgatcaccgacgagtacaaggtgccaagcaagaagttcaaggtgctgggcaacaccgaccgtcactccatcaagaagaacctcatcggcgccctcctcttcgactcaggtgagacggccgaggcgacaaggttgaagaggacggcaagaagaaggtacacaagaaggaagaacaggatctgctacctccaggagatcttcagcaacgagatggccaaggtggatgacagcttcttccaccggctagaggagagcttcttggtggaggaggacaagaagcatgagaggcatcccatcttcggcaacatcgtcgacgaggtggcctaccatgagaagtaccccaccatctaccatctccgcaagaagctggtggactcaacggacaaggccgacctccgcctcatctacctcgccttggcccacatgatcaagttcagaggccacttcctcatcgagggcgacctcaaccctgacaacagcgatgtggacaagctcttcatccagctggtgcagacctacaaccagctctttgaggagaaccccatcaacgcctccggcgtcgacgccaaggccatcctctcggcaaggttgagcaagtcaagaaggctggagaacctcatcgctcagctccccggcgagaagaaaaatggcctcttcggcaacctcatcgccttgagcctcggcctcacccccaacttcaagtcaaactttgacctcgccgaggatgccaagctccagctctccaaggacacctacgacgacgacctcgacaacctcctcgcccagatcggcgaccagtacgccgacctcttcctcgccgccaagaacctctcagatgccatcctcctgagcgacatcctccgcgtcaacaccgagatcaccaaggcgccgctctccgcctccatgatcaagaggtatgatgagcaccatcaagatctcaccctcctcaaggccttggtgaggcagcagctacctgagaagtacaaggagatcttcttcgaccagagcaagaatggctacgccggctacatcgacggcggcgcctcccaagaagagttctacaagttcatcaagcccatcttggagaagatggatggcaccgaggagctactggtgaagctcaacagagaagatctcctcaggaagcagaggaccttcgacaacggcagcatccctcaccagatccaccttggtgagctccatgccatcttgagaaggcaagaagacttctaccccttcctcaaggacaacagggagaagatcgagaagatattgacattcaggatcccctactacgtcggacccttggcaagaggcaactcaaggttcgcctggatgacaaggaagagcgaggaaaccatcaccccctggaactttgaggaggtggtggacaagggcgcctcagctcagagcttcatcgagaggatgaccaacttcgacaagaacctccccaacgagaaggtgctccccaagcacagcctcctctacgagtacttcaccgtctacaatgagctcaccaaggtcaagtacgtcaccgagggcatgaggaagccggccttcctctcaggagagcagaagaaggccatcgtcgacctcctcttcaagaccaacaggaaggtcaccgtcaagcagctcaaggaggactacttcaagaagatcgagtgcttcgactctgtggagatctccggcgtggaggacaggttcaacgcctccctcggcacctaccatgatctcctgaagatcatcaaggacaaggacttcctcgacaacgaggagaacgaggacatcttagaagacatcgtcctcaccctcaccctcttcgaggacagggagatgatcgaggagaggctcaagacctatgctcacctctttgatgacaaggtgatgaagcagctcaagagaagaaggtacaccggctggggccgcctctcaaggaagctcatcaacggcatcagggacaagcagagcggcaagaccatcctcgacttcctcaagagcgatggcttcgccaacaggaacttcatgcagctcatccatgatgacagcttgaccttcaaggaggacatccagaaggctcaagtctccggccaaggagattccctccatgagcacatcgccaacctcgccggctcaccggccatcaagaagggcatcctccagaccgtcaaggtggtggatgagctggtgaaggtgatgggccgtcacaagccggagaacatcgtcatcgagatggcaagggagaaccagaccacccagaagggccagaagaactcaagggagaggatgaagaggattgaagaaggcatcaaggagctcggcagccagatcctcaaggagcaccctgtggagaacacccagctacagaacgagaagctctacctctactacctccagaatggccgtgacatgtacgtcgaccaagagctggacatcaacaggttgagcgactacgacgtcgaccacatcgtcccccagagcttcctcaaggatgacagcatcgacaacaaggtgctgacaagatcagacaagaaccgtggcaagagcgacaatgtcccctccgaggaggtggtgaagaagatgaagaactactggaggcagctcctcaacgccaagctcatcacccagaggaagttcgacaacctcaccaaggctgaaagaggaggcctctcagagctggacaaggccggcttcatcaagaggcagctggtggagacaaggcagatcaccaagcatgttgctcagatccttgattcaaggatgaacaccaagtacgacgagaacgacaagctcatcagggaggtcaaggtcatcaccctcaagagcaagctggtgtccgacttcaggaaggacttccagttctacaaggtgagggagatcaacaactaccaccatgctcatgatgcctacctcaacgccgtggtgggcaccgccctcatcaagaagtaccccaagctggagtcagagttcgtctacggcgactacaaggtgtatgatgtgaggaagatgatcgccaagagcgagcaggagatcggcaaggccaccgccaagtacttcttctactcaaacatcatgaacttcttcaagaccgagatcaccctcgccaacggcgagatcaggaagaggccgctcattgagacaaatggtgagactggtgagatcgtctgggacaagggccgagacttcgccaccgtgaggaaggtgctctccatgcctcaagtcaacatcgtcaagaagaccgaggtgcagaccggaggcttctcaaaggagagcatcctccccaagaggaactctgacaagctgatcgcaaggaagaaggactgggaccccaagaagtacggcggctttgattcaccaacggtggcctactccgtgctggtggtggccaaggtggagaagggcaagagcaagaagctcaagagcgtcaaggagctcctcggcatcaccatcatggagagatcatccttcgagaagaaccccatcgacttccttgaggccaagggctacaaggaggtgaagaaggacctcatcatcaagctccccaagtacagcctcttcgagctggagaatggccgcaagaggatgctggcatcagctggagagctccagaagggcaacgagctggccctcccaagcaagtacgtgaacttcctctacctcgcctcccactacgagaagctcaagggctcaccagaagacaatgagcagaagcagctcttcgtggagcagcacaagcactacctcgacgagatcatcgagcagatctccgagttctccaagagggtgatcctcgccgacgccaacctcgacaaggtgctctcagcctacaacaagcaccgtgacaagcccatcagggagcaagctgagaacatcatccacctcttcaccctcaccaacctcggcgccccggcggccttcaagtacttcgacaccaccatcgaccgcaagaggtacacctcaaccaaggaggtgcttgatgccaccttgatccaccagagcatcaccggcctctacgagacaaggatcgacctctcccagctcggcggcgacaagaggccggcggccaccaagaaggccggccaagccaagaagaagaagtgattgctagcacgcgttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcggcgcgccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - protein, 1401 aa, Artificial Sequence
  Cas9 D10A nickase from Plasmid BAY02734
MAPKKKRKVGIHGVPAAMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 5 - DNA, 3120 bp, Artificial Sequence
  Plasmid BAY02528 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaacttaattaaaaaaaaaaagcaccgactcggtgccactttttcaagttgataacggactagccttattttaacttgctatttctagctctaaaactgtatgttctccacacctagcaagtctgatgcagcaagcgagtttataaaccggtcagaactctgctggacaggcgaggtgggactaaactaagaggaactgctgcgttgctctccccaatcgtgaacacgtcgaccacccgacaaaagcccgatcatgcgggcctaaaagcccagcgctagaccgttgctgaactcacgtgggctgcatctcactatgcgacccagatcagtgagagctagagggcagttatcttcagctctctcgtagttcctctgatcaatattcctagttcgattccctcctagcagtgagtaacaatgtgctccttgataaatataaatgtgttgagcaccagaactgtcagaataacgggcttggtccctgcagggttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 6 - DNA, 3119 bp, Artificial Sequence
  Plasmid BAY02529 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaacttaattaaaaaaaaaaagcaccgactcggtgccactttttcaagttgataacggactagccttattttaacttgctatttctagctctaaaaccacctagcccatcctccttccaagtctgatgcagcaagcgagtttataaaccggtcagaactctgctggacaggcgaggtgggactaaactaagaggaactgctgcgttgctctccccaatcgtgaacacgtcgaccacccgacaaaagcccgatcatgcgggcctaaaagcccagcgctagaccgttgctgaactcacgtgggctgcatctcactatgcgacccagatcagtgagagctagagggcagttatcttcagctctctcgtagttcctctgatcaatattcctagttcgattccctcctagcagtgagtaacaatgtgctccttgataaatataaatgtgttgagcaccagaactgtcagaataacgggcttggtccctgcagggttcatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 3117 bp, Artificial Sequence
  Plasmid BAY02530 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggataaccctgcagggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgataggccctagaataggcacgttttagagctagaaatagcaagttaaaataaggctagtccgttatcaacttgaaaaagtggcaccgagtcggtgctttttttttttaattaagttcatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 3117 bp, Artificial Sequence
  Plasmid BAY02531 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaaccctgcagggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgctcctcataggccctagaatgttttagagctagaaatagcaagttaaaataaggctagtccgttatcaacttgaaaaagtggcaccgagtcggtgctttttttttttaattaagtatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 3119 bp, Artificial Sequence
  Plasmid BAY02532 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaaccctgcagggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgctattgccagtgcctattctgttttagagctagaaatagcaagttaaaataaggctagtccgttatcaacttgaaaaagtggcaccgagtcggtgctttttttttttaattaagttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 3119 bp, Artificial Sequence
  Plasmid BAY02524 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaacttaattaaaaaaaaaaagcaccgactcggtgccactttttcaagttgataacggactagccttattttaacttgctatttctagctctaaaacgacttgcccagcacgaggaacaagtctgatgcagcaagcgagtttataaaccggtcagaactctgctggacaggcgaggtgggactaaactaagaggaactgctgcgttgctctccccaatcgtgaacacgtcgaccacccgacaaaagcccgatcatgcgggcctaaaagcccagcgctagaccgttgctgaactcacgtgggctgcatctcactatgcgacccagatcagtgagagctagagggcagttatcttcagctctctcgtagttcctctgatcaatattcctagttcgattccctcctagcagtgagtaacaatgtgctccttgataaatataaatgtgttgagcaccagaactgtcagaataacgggcttggtccctgcagggttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 3120 bp, Artificial Sequence
  Plasmid BAY02533 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaaccctgcagggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttggaacaaccagcatctgggaagttttagagctagaaatagcaagttaaaataaggctagtccgttatcaacttgaaaaagtggcaccgagtcggtgctttttttttttaattaagttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 3120 bp, Artificial Sequence
  Plasmid BAY02535 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaaccctgcagggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgatctgggaatggtggtgcaggttttagagctagaaatagcaagttaaaataaggctagtccgttatcaacttgaaaaagtggcaccgagtcggtgctttttttttttaattaagttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 13 - DNA, 3455 bp, Artificial Sequence
  Plasmid BAY02539 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaacggcgcgccatgtattctcagggatgtaagcaacgggtctgtcaggtgggtccaaagattttgtaataggaagaggtccaccaatgttggcaggaacatagctgagccacctcaatatattagatacaccttcaaggtcatctgaaactgtcagatggacaacaccgtttgtcgccataattttggggccacccaactgcatgtgggagctgtaaacttcccggccaagaagcttgttcaaggcagagaacccagttaggataatgggctggtcagtacgctgtatgcaccgtatgccaagtcgagcaagatatgctcctattccaacagtccgtccagtcacaaatgtaagcgtaaatgtctcctcataggccctagaataggcactggcaatagcagcacttccatggaggttctcgacgcccaacccatcctccttccccacaacagaatcgataacccacctaatttcaccattatcaagctgcatcttgtgcgctataacagaagtgctaatacgagcatggtcttcttcagtcagataaatgtactgaaacccacgttcagggctgccatcatcagaccatccaacacggaagcaagattttacttcatctgctatgccgatccgagcaccagagtttgctgccaagtagatgagaggaagcttcctctcacaagctaggttggtaacagtttcaaaaaatgcatcttcccttggaccaaacgatccagctctaaaagtaatatcatttgcgatgacaacaatctgcctgccattgggatattcaggagtggacatgtccaagatcttaattaagttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 3455 bp, Artificial Sequence
  Plasmid BAY02540 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaacttaattaagatcttggacatgtccactcctgaatatcccaatggcaggcagattgttgtcatcgcaaatgatattacttttagagctggatcgtttggtccaagggaagatgcattttttgaaactgttaccaacctagcttgtgagaggaagcttcctctcatctacttggcagcaaactctggtgctcggatcggcatagcagatgaagtaaaatcttgcttccgtgttggatggtctgatgatggcagccctgaacgtgggtttcagtacatttatctgactgaagaagaccatgctcgtattagcacttctgttatagcgcacaagatgcagcttgataatggtgaaattaggtgggttatcgattctgttgtggggaaggaggatgggctaggtgtggagaacctccatggaagtgctgctattgcgagtgcgtatagccgcgcctatgaggagacatttacgcttacatttgtgactggacggactgttggaataggagcatatcttgctcgacttggcatacggtgcatacagcgtactgaccagcccattatcctaactgggttctctgccttgaacaagcttcttggccgggaagtttacagctcccacatgcagttgggtggccccaaaattatggcgacaaacggtgttgtccatctgacagtttcagatgaccttgaaggtgtatctaatatattgaggtggctcagctatgttcctgccaacattggtggacctcttcctattacaaaatctttggacccacctgacagacccgttgcttacatccctgagaatacatggcgcgccgttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 3455 bp, Artificial Sequence
  Plasmid BAY02541 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaacggcgcgccatgtattctcagggatgtaagcaacgggtctgtcaggtgggtccaaagattttgtaataggaagaggtccaccaatgttggcaggaacatagctgagccacctcaatatattagatacaccttcaaggtcatctgaaactgtcagatggacaacaccgtttgtcgccataattttggggccacccaactgcatgtgggagctgtaaacttcccggccaagaagcttgttcaaggcagagaacccagttaggataatgggctggtcagtacgctgtatgcaccgtatgccaagtcgagcaagatatgctcctattccaacagtccgtccagtcacaaatgtaagcgtaaatgtctcctcataggcgcggctatacgcactcgcaatagcaacacttccatggaggttctccacacctagcccatcctccttccccacaacagaatcgataacccacctaatttcaccattatcaagctgcatcttgtgcgctataacagaagtgctaatacgagcatggtcttcttcagtcagataaatgtactgaaacccacgttcagggctgccatcatcagaccatccaacacggaagcaagattttacttcatctgctatgccgatccgagcaccagagtttgctgccaagtagatgagaggaagcttcctctcacaagctaggttggtaacagtttcaaaaaatgcatcttcccttggaccaaacgatccagctctaaaagtaatatcatttgcgatgacaacaatctgcctgccattgggatattcaggagtggacatgtccaagatcttaattaagttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 3439 bp, Artificial Sequence
  Plasmid BAY02536 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaacggcgcgccggagctttgccttcagatttatcaattctggatcaagcctacccatgcactcttggagttcctctgacctgaacttgatctcgatcaacccttgaggttcgagaacattgccctttgcagtcctctcagcatagaactcaatgcgatctggatttatcttgctatcaatcacgacccaagcccctccacgtagctctgcagccttggggatatatacaaaggcaggctgattgtatgtcctaaggttctcaacaattgttgacccagcctgaaggattccttcaaaaagatctctttgtccaccagagaagcctctccagttagcaaggatgaacagaggtaatccttcacggttgaagtccagcattgcctgcgctgtcttcgtgacgctgtccgggaaccacacctgcccagcacgaggaacagatcgctcatgggaatcaagctggcctggatcagcagggatgagctgcatcatagtctgtgtctccacagctataacacccaccggaatccctccgagtttcgctctgccagtaacaactgacttcgcccatccttcaaatgtctccacaaaactgtctttgtcgaacatgccccccaaccatttcccttggctatcatcaatgccactgatggcagcacgaggatcgcatgtattctcagggatgtaagcaacgggtctgtcaggtgggtccaaagattttgtaataggaagaggtccaccaatgttggcaggaacatagctgagccacctcaatatattagatacaccttcaaggtcatcttaattaagttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 3457 bp, Artificial Sequence
  Plasmid BAY02542 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaacttaattaaggtccatggcacaaggagttggatcagcagaagagggagtttcctctaggattcaagacttttggcgaggccatcccgccgcaatatgctatccaggtactggatgagctgacaaaaggggaggcgatcattgccactggtgttgggcagcaccagatgtgggcggctcagtattacacttacaagcggccacggcagtggctgtcttcgtctggtttgggggcaatgggatttgggttaccagctgcagctggcgctgctgtggccaacccaggtgttacagttgttgacattgatggtgatggtagtttcctcatgaacattcaggagttggcgttgatccgcattgagaacctcccagtgaaggtgatgatattgaacaaccagcatctcggcatggtcgtccagctcgaggataggttttacaaggccaatcgggcgcacacataccttggcaacccagaaaatgagagtgagatatatccagattttgtgacgattgctaaaggattcaacgttccagcagttcgagtgacgaagaagagcgaagtcactgcagcaatcaagaagatgcttgagaccccagggccatacttgttggatatcatagtcccgcatcaggagcacgtgctgcctatgatcccaagcggtggtgctttcaaggacatgatcatggagggtgatggcaggacctcgtactgaaatttcgacctacaagacctacaagtgtgacatgcgcaatcagcatgatgcccgcgtgttgtatcaactactaggggttcaactgtgagccatgcgttttctaggcgcgccgttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 18 - DNA, 6635 bp, Artificial Sequence
  Plasmid IB26 (transforming DNA)
gtgcaccatacctgcaggcaattggccgctgtaccatgcatgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaatagagagagatagatttgtagagagagactggtgatttcagcgtgtccaagcttgctagagtcgcggccgcttcagatctcggtgacgggcaggaccggacgtggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatcttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggttttggtttaataagaagagaaaagagttcttttgttatggctgaagtaatagagaaatgagctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcaggggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatgtatggtgcatatggcgcgccatagatccgtcaattctcactcattaggcaccccaggctttacactttatgcttccggctcgtataatgtgtggaattgtgagcggataacaatttcacacaggaaacaggatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatccggcccatgatcatgtggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccctgtacagcggccgcgttaacgcgtatactctagagcgatcgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgaga

SEQ ID NO: 19 - DNA, 19 bp, Artificial Sequence
  Primer HT-18-048
atgatggcagccctgaacg

SEQ ID NO: 20 - DNA, 20 bp, Artificial Sequence
  Primer HT-18-051
ggcatgttcgacaaagacag

SEQ ID NO: 21 - DNA, 19 bp, Artificial Sequence
  Primer HT-18-053
caactgcatgtgggagctg

SEQ ID NO: 22 - DNA, 20 bp, Artificial Sequence
  Primer HT-18-054
acaattgttgacccagcctg

SEQ ID NO: 23 - DNA, 23 bp, Artificial Sequence
  Primer HT-18-065
gttacagttgttgacattgatgg

SEQ ID NO: 24 - DNA, 20 bp, Artificial Sequence
  Primer HT-18-066
aagtatggccctggggtctc

SEQ ID NO: 25 - DNA, 23 bp, Artificial Sequence
  Primer HT-18-101
ctattgccagtgcctattctagg

SEQ ID NO: 26 - DNA, 21 bp, Artificial Sequence
  Primer HT-18-102
gtgacgctgtccgggaaccac

SEQ ID NO: 27 - DNA, 20 bp, Artificial Sequence
  Primer HT-18-110
ctgcttgccacttacacctc

SEQ ID NO: 28 - DNA, 19 bp, Artificial Sequence
  Primer HT-18-111
gttgggcgtcgagaacctc

SEQ ID NO: 29 - DNA, 20 bp, Artificial Sequence
  Primer HT-18-112
acttgcccagcacgaggaac

SEQ ID NO: 30 - DNA, 19 bp, Artificial Sequence
  Primer HT-18-113
gctaggtgtggagaacctc

SEQ ID NO: 31 - DNA, 21 bp, Artificial Sequence
  Primer HT-18-130
gatgcatggcactgtgtatgc

SEQ ID NO: 32 - DNA, 18 bp, Artificial Sequence
  Primer HT-18-135
ggcatggtcgtccagctc

SEQ ID NO: 33 - DNA, 21 bp, Artificial Sequence
  Primer HT-18-136
cagaaaggaaccagcacactg

SEQ ID NO: 34 - DNA, 18 bp, Artificial Sequence
  Primer HT-18-162
gttccttccccttgcatc

SEQ ID NO: 35 - DNA, 3496 bp, Artificial Sequence
  Plasmid Bas04096 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggcttttggcggaatgatcaattgccctcgtttattgcaccgctgatttccggtgggcatattaaccgatcttggacatgtccactcctgaatatcccaatggcaggcagattgttgtcatcgcaaatgatattacttttagagctggatcgtttggtccaagggaagatgcattttttgaaactgttaccaacctagcttgtgagaggaagcttcctctcatctacttggcagcaaactctggtgctcggatcggcatagcagatgaagtaaaatcttgcttccgtgttggatggtctgatgatggcagccctgaacgtgggtttcagtacatttatctgactgaagaagatcatgctcgtattagcacttctgttatagcgcacaagatgcagcttgataatggtgaaatccgctgggttatcgattctgttgtggggaaggaggatgggctaggtgtggagaacctccatggaagtgctgctattgccagtgcctattctagggcctatgaggagacatttacgcttacatttgtgactggacggactgttggaataggagcatatcttgctcgacttggcatacggtgcatacagcgtactgatcagcccattatcctaactgggttctctgccttgaacaagcttcttggccgggaagtttacagctcccacatgcagttgggtggccccaaaattatggcgacaaacggtgttgtccatctgacagtttcagatgaccttgaaggtgtatctaatatattgaggtggctcagctatgttcctgccaacattggtggacctcttcctattacaaaatctttggacccacctgacagacccgttgcttacatccctgagaatacatatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 36 - DNA, 3490 bp, Artificial Sequence
  Plasmid Bay02544 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatccaagctcggcgcgccattgggatggcgcgccatgtattctcagggatgtaagcaacgggtctgtcaggtgggtccaaagattttgtaataggaagaggtccaccaatgttggcaggaacatagctgagccacctcaatatattagatacaccttcaaggtcatctgaaactgtcagatggacaacaccgtttgtcgccataattttggggccacccaactgcatgtgggagctgtaaacttcccggccaagaagcttgttcaaggcagagaacccagttaggataatgggctggtcagtacgctgtatgcaccgtatgccaagtcgagcaagatatgctcctattccaacagtccgtccagtcacaaatgtaagcgtaaatgtctcctcataggcgcggctatacgcactcgcaatagcaacacttccatggaggttctcgacgcccaacccatcctccttccccacaacagaatcgataacccacctaatttcaccattatcaagctgcatcttgtgcgctataacagaagtgctaatacgagcatggtcttcttcagtcagataaatgtactgaaacccacgttcagggctgccatcatcagaccatccaacacggaagcaagattttacttcatctgctatgccgatccgagcaccagagtttgctgccaagtagatgagaggaagcttcctctcacaagctaggttggtaacagtttcaaaaaatgcatcttcccttggaccaaacgatccagctctaaaagtaatatcatttgcgatgacaacaatctgcctgccattgggatattcaggagtggacatgtccaagatcttaattaaatcccaatacgcgtcaattcaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 37 - DNA, 3155 bp, Artificial Sequence
  Plasmid Bas04093 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgatgagaccgaggtcgtgatgtctgaagtacgccaatacatctatggcatcggtacgcagtgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgacccagttaggataatgggcgttttagagctagaaatagcaagttaaaataaggctagtccgttatcaacttgaaaaagtggcaccgagtcggtgctttttttttatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 38 - DNA, 8853 bp, Artificial Sequence
  Plasmid Bas03568 encoding a modified LbCas12a
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgggcccggccggccgcgatcgcgcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagccatggcgcccaagaagaagaggaaggtgggcatccacggcgtccccgccgccatgagcaagctggagaagttcaccaactgctactcactctccaagaccctccgcttcaaggccatccccgtcggcaagacccaggagaacatcgacaacaagaggctgctggtggaggatgagaagagggccgaggactacaagggcgtcaagaagctcctcgaccggtactacctctcattcatcaatgatgtcctccactccatcaagctgaagaacctcaacaactacatctccctcttccgcaagaagacaaggaccgagaaggagaacaaggagctggagaacctggagatcaacctccgcaaggagatcgccaaggccttcaagggcaacgagggctacaagagcctcttcaagaaggacatcatcgagacgatcctaccggagttcctggatgacaaggacgagatcgccctggtgaacagcttcaacggcttcaccaccgccttcaccggcttcttcgacaaccgggagaacatgttctccgaggaggccaagagcacctccatcgccttcaggtgcatcaacgagaacctcacccgctacatctcaaacatggacatcttcgagaaggtggacgccatcttcgacaagcatgaggtccaggagatcaaggagaagatcctgaactccgactacgacgtggaggacttcttcgagggcgagttcttcaacttcgtcctgacccaagaaggcatcgacgtctacaacgccatcatcggcggcttcgtcaccgagtcaggtgagaagatcaagggcctcaacgagtacatcaacctctacaaccagaagaccaagcagaagctccccaagttcaagcccctctacaagcaggtgctctccgaccgtgagagcttgagcttctacggcgagggctacacctccgacgaggaggtgctggaggtcttccgcaacaccctcaacaagaactcagagatcttctcctccatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcactggagcagctccaggagtacgccgacgccgacctctccgtggtggagaagctgaaggagatcatcatccagaaggtggacgagatctacaaggtgtacggcagctctgagaagctcttcgacgccgacttcgtcctggagaagagcctcaagaagaacgacgccgtggtggccatcatgaaggacctcctcgactcagtcaagagcttcgagaactacatcaaggccttcttcggcgagggcaaggagacaaaccgcgacgagagcttctacggcgacttcgtcctcgcctatgacatcctcctcaaggtggaccacatctacgacgccatcaggaactacgtcacccagaagccctactcaaaggacaagttcaagctctacttccagaacccccagttcatgaggggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctaccaagggtcttcttctccaagaagtggatggcctactacaacccctccgaggacatccagaagatctacaagaacggcaccttcaagaagggtgacatgttcaacctcaacgactgccacaagctgatcgacttcttcaaggacagcatctcaaggtaccccaagtggagcaacgcctacgacttcaacttctctgagacggagaagtacaaggacatcgccggcttctaccgggaggtggaggagcaaggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggagggcctccctcaagaaggaggagctggtggttcacccggccaacagccccatcgccaacaagaaccctgacaaccccaagaagaccaccaccctctcctacgacgtctacaaggacaagaggttctcagaagaccagtacgagctccacatccccatcgccatcaacaagtgccccaagaacatcttcaagatcaacaccgaggtgagggtgctactcaagcatgatgataacccctacgtcatcggcatcgaccgcggcgagaggaacctcctctacatcgtggtggtggacggcaagggcaacatcgtggagcagtactccttgaacgagatcatcaacaacttcaacggcatcaggatcaagaccgactaccactcactccttgacaagaaggagaaggagaggttcgaggcccggcaaaattggacaagcatcgagaacatcaaggagctgaaggccggctacatctcccaggtggtgcacaagatctgtgagctggtggagaagtacgacgccgtcatcgccctggaggacctcaacagcggcttcaagaactcaagggtcaaggtggagaagcaggtgtaccagaagttcgagaagatgctcatcgacaagctgaactacatggtggacaagaagagcaacccctgcgccaccggcggcgccctcaagggctaccagatcaccaacaagtttgagagcttcaagagcatgtcaacccagaacggcttcatcttctacatcccggcctggctcacctccaagatcgacccctccaccggcttcgtcaacctcctcaagaccaagtacacctccatagccgacagcaagaagttcatctcctccttcgaccgcatcatgtacgtccctgaggaggacctcttcgagttcgccctcgactacaagaacttctcaaggaccgacgccgactacatcaagaagtggaagctctacagctacggcaaccgcatcaggatcttcaggaaccccaagaagaacaacgtcttcgactgggaggaggtgtgcctgacctccgcctacaaggagctcttcaacaagtacggcatcaactaccagcaaggtgacatcagggcgctcctatgcgagcagagcgacaaggccttctactcctccttcatggccctcatgagcttgatgctccagatgaggaacagcatcaccggccgcaccgacgtcgacttcctcatctcccccgtcaagaactctgatggcatcttctacgactcaaggaactacgaggcccaggagaacgccatcctccccaagaacgccgacgccaacggcgcctacaacatcgccaggaaggtgctctgggccatcggccagttcaagaaggccgaggatgagaagctggacaaggtgaagatcgccatctcaaacaaggagtggctggagtacgcccagacctccgtcaagcacaagaggccggcggccaccaagaaggccggccaggccaagaagaagaagggcagctacccctacgacgtccccgactacgcctacccctacgacgtccccgactacgcctacccctacgacgtccccgactacgcctaattgctagcacgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggcccgggttaattaaatttaaatggcgcgccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 39 - protein, 1290 aa, Artificial Sequence
  Modified LbCas12a from Plasmid Bas03568
MAPKKKRKVGIHGVPAAMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYA

SEQ ID NO: 40 - DNA, 20 bp, Artificial Sequence
  Primer HT-19-022
ggctaggagtcgaaaacctc

SEQ ID NO: 41 - DNA, 2918 bp, Artificial Sequence
  Plasmid Bas03609 comprising guide RNA
gcgggtgttggcgggtgtcggggctggcttaactatgcggcatcagagcagattgtactgagagaaacccaattgggtaccgagctccctgcaggaagcttaattaagatgagaccgaggtcgtgatgtctgaagtacgccaatacatctatggcatcggtacgcagtgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgtaatttctactaagtgtagatcacctagcccatcctccttccccttttattttttggcgcgccacctgctagcgctcgcttggatccgaattcaaacctgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtca

SEQ ID NO: 42 - DNA, 3455 bp, Artificial Sequence
  Plasmid Bas03253 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaacggcgcgccatgtattctcagggatgtaagcaacgggtctgtcaggtgggtccaaagattttgtaataggaagaggtccaccaatgttggcaggaacatagctgagccacctcaatatattagatacaccttcaaggtcatctgaaactgtcagatggacaacaccgtttgtcgccataattttggggccacccaactgcatgtgggagctgtaaacttcccggccaagaagcttgttcaaggcagagaacccagttaggataatgggctggtcagtacgctgtatgcaccgtatgccaagtcgagcaagatatgctcctattccaacagtccgtccagtcacaaatgtaagcgtaaatgtctcctcataggccctagaataggcactggcaatagcagcacttccatggaggttttcgactcctagcccatcctccttccccacaacagaatcgataacccacctaatttcaccattatcaagctgcatcttgtgcgctataacagaagtgctaatacgagcatggtcttcttcagtcagataaatgtactgaaacccacgttcagggctgccatcatcagaccatccaacacggaagcaagattttacttcatctgctatgccgatccgagcaccagagtttgctgccaagtagatgagaggaagcttcctctcacaagctaggttggtaacagtttcaaaaaatgcatcttcccttggaccaaacgatccagctctaaaagtaatatcatttgcgatgacaacaatctgcctgccattgggatattcaggagtggacatgtccaagatcttaattaagttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 43 - DNA, 9186 bp, Artificial Sequence
  Plasmid KVA824 encoding Cas9 D10A nickase
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgggcccggccggccgcgatcgcgcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagccatggcccctaagaagaaacgaaaggtaggcatccacggtgttccagcggctatggacaagaagtacagcatcgggctggccattggcacgaactctgttgggtgggcggttatcactgacgagtacaaggtgccgtcgaagaagttcaaggtgctaggcaacaccgaccgtcacagcatcaagaagaacctcatcggcgccctgttgtttgactctggggaaaccgccgaggctactcgactaaagagaaccgcccgtaggcgatacactaggcggaagaacaggatctgttatctccaggagatcttcagcaacgagatggcgaaggtcgatgactcattcttccaccgccttgaggagtctttcctggtcgaggaggacaaaaagcacgagcgacacccgatcttcgggaacatcgttgatgaggtagcctaccacgagaagtaccctactatctaccacctgcgcaagaaactagtggactccaccgacaaggcggacctccgccttatctatctcgccctcgcccacatgatcaagtttcgcgggcacttcctcatcgaaggcgatctcaatccggacaacagcgacgtggacaagctattcatccagctagttcagacctacaaccagctctttgaagagaaccctattaacgcgagcggggtggacgccaaagccatcctttccgccaggctctctaagagccggagactcgagaaccttatcgctcagttgcccggcgagaagaaaaacggccttttcggcaacctgattgccctgtccttgggcttgacgccgaacttcaagagcaacttcgacctagctgaggatgctaaactgcagctgtccaaggacacttatgatgacgaccttgacaacctcctggcccaaattggggatcagtacgcggacctcttcctcgctgccaagaacctctccgacgcgattctgttgtcggacatcctccgcgtgaacaccgagattactaaggccccactgtccgcgagcatgatcaaacgctacgacgagcaccatcaggatctaactctgctcaaggcgctcgtccgtcagcaactccctgagaagtacaaggagatcttcttcgaccagtccaagaacggctacgccggttacattgatggtggcgcgagccaggaggagttctacaagttcatcaagcccatcttagagaagatggacggcaccgaagagcttctcgtgaagctcaacagggaggacctcctaaggaagcagcgcacctttgacaacgggtctattcctcatcagatccatctcggcgagctccacgcgattttgagacgccaagaggacttctacccgttcttgaaggacaaccgggagaagatcgagaagatcctcacgttcaggatcccttattacgtcggccccctagctaggggtaactcgagattcgcgtggatgaccagaaagtccgaggaaaccattaccccgtggaactttgaagaggtggtcgacaagggcgcgagcgcgcaatcgttcattgagcggatgaccaacttcgacaagaacctaccgaacgagaaggtcctgccgaaacactccctgctctacgagtacttcaccgtttataacgagctgacgaaggtcaagtacgtgactgagggcatgcgtaaaccagcgttcctcagcggtgagcagaagaaggctatcgtggacctgctctttaagacgaaccgcaaggtgaccgtgaagcagctcaaggaggattatttcaagaagatcgagtgcttcgactcagtggagattagcggcgtggaggaccgtttcaatgcgagcttaggtacctaccacgacctcctgaagatcatcaaggacaaggacttcctcgacaacgaggaaaacgaggacatcctggaggacatcgtgctcaccctgaccctctttgaagacagggagatgatcgaggaaaggttgaagacctacgcgcacctattcgacgacaaggtcatgaaacagctcaagcggaggagatacactggctggggcagactttcgcggaagctcatcaatgggatcagggacaagcagtccggcaagaccatcctggacttcttgaagagcgacggcttcgctaatcgcaacttcatgcagctcattcatgacgactccctaacattcaaggaggacatccaaaaggcgcaagtctcgggtcagggggattctttgcacgagcacatcgcaaacctcgccggttctccggccattaaaaagggcatccttcagacggtcaaggtggtcgacgagctcgtgaaggttatgggccggcataagcccgagaacatagtgatcgagatggctagggagaaccagaccacgcaaaagggccagaagaactcacgtgagcgcatgaagaggatcgaggaaggtatcaaggagctgggctctcagattctcaaggagcatccggtcgagaacacccagcttcagaacgagaagctgtacctctactacctccagaatggccgcgacatgtacgttgatcaggagttagatattaatcggctgagcgactacgacgttgaccacatcgtgccccagtcgttcctcaaggatgactccatcgacaacaaggtcctgaccagatcggacaagaaccgtggtaagagcgacaatgtgccctccgaggaggtggtcaaaaagatgaagaactattggcggcagttgctgaacgccaagctcatcacccagcgaaagttcgataatttaacgaaggccgaacgagggggcctttccgaactcgataaggccggtttcatcaagcgtcagctggttgagacgcgacagatcacgaagcatgtggcccagattctggacagccggatgaacacgaagtacgacgagaacgacaaacttattcgggaggtgaaggtgataaccctcaagtccaagttagtgtcggacttccgcaaggactttcagttctataaagtgcgcgagatcaacaattaccaccacgcccacgacgcctacttgaatgccgtggtcggcacggcgctcatcaaaaagtaccccaagctggagagcgagttcgtctacggcgactacaaggtttatgacgtcaggaagatgattgcgaagtcagagcaagagatcgggaaggccaccgccaagtacttcttctactcgaacatcatgaacttctttaagaccgagatcaccctcgccaatggggagatccggaaacgtccccttatcgagacgaacggcgagactggcgagatagtctgggacaaaggtcgcgatttcgccaccgttcgaaaggtgctctcaatgcctcaggtcaatatcgtgaagaagaccgaggttcagacggggggcttcagcaaggagtccatcctccctaagcgcaactctgacaagctgatcgcccggaaaaaggattgggacccgaaaaagtacggcggcttcgattctcccaccgtagcttatagcgtgctcgtggttgccaaggtggagaagggcaagagcaagaaactgaagtcggtcaaggagttactcggcatcacaatcatggagcgcagctcgtttgaaaagaacccgatcgacttcctggaggccaagggctataaggaggtgaagaaggacctcatcattaagctgccgaagtacagcttgttcgagctcgagaatggcaggaagaggatgctagcttcggccggcgaacttcaaaaagggaacgagctcgcgttgccgtcgaagtacgtgaacttcctctacttagctagtcactatgaaaagctgaagggctcgccggaggacaacgagcagaagcagctcttcgtcgagcaacacaagcactacctcgacgagatcatcgagcagataagtgagttcagcaagcgcgtgatcctcgctgatgccaacctcgataaggtgctcagcgcctacaataagcaccgagacaagccgattcgggagcaggcggagaacatcattcatttatttacgctcactaacctaggcgctccggctgccttcaagtacttcgacaccacgatcgacaggaagcgctacaccagcaccaaagaagtcctcgacgcgacgctcattcaccagagcatcacagggctctacgagactcggatcgacttgtcgcaattgggtggggataagaggccggccgcgactaaaaaggccggccaagccaagaaaaagaaatgattgctagcacgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggcccgggttaattaaatttaaatggcgcgccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 44 - protein, 1401 aa, Artificial Sequence; identical to SEQ ID NO: 4
  Cas9 D10A nickase from Plasmid KVA824

SEQ ID NO: 45 - DNA, 3318 bp, Artificial Sequence
  Plasmid KVA766 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaaccctgcaggaaaaaaaaagcaccgactcggtgccactttttcaagttgataacggactagccttattttaacttgctatttctagctctaaaacttgacagcagccgtgactgcaagctgactacagcgcgcgggtttataagctggttccatcgtctttcggtttccgatgtggtactaaacatcccccgcaacgccaccgcgcaaaccatcggatccccatccaacggactaaagtgggtagcagcgccacccgcgcggtacggcgcgcgtgggctgcaccttccgacgcagcccaaatcaggatacggcccattagtatatattctcctcctcccagcccacatcacatgcgtttcacactttcgcctgtaatcgctcaagtagtcaagtatgagctctgtctgcacgaactgattgtcactagtgtaccatcaaactactagacgtgacttgtgataatctcagacgcttatggagtgaaacatggtaaaaaaatgcgtttatgtgcttgatcactttgatcagccttgcctccatcgtctgaaacatgataagaagacctttgttacgaaagtgtgaaacgattctgttattccttcggctatgccgtaattcaactttcacaaagcgatcgcgttccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 46 - DNA, 3349 bp, Artificial Sequence
  Plasmid KVA769 comprising guide RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgttgtaaaacgacggccagtgcgatcgctttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgaggaagtgcaactcttcttggttttagagctagaaatagcaagttaaaataaggctagtccgttatcaacttgaaaaagtggcaccgagtcggtgctttttttttcctgcaggggcgtaatcatggtcatagcatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 47 - DNA, 3645 bp, Artificial Sequence
  Plasmid KVA791 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcgatcgcttctcccaaaattatgcaattttgaggctcctctacatcattataattccccaatacattgctctttattcttaacagctttgatcgcgaaatttaacattttaattcttgagctgttattttgtagcatcagtttatcatgagccatgtttggtactaaatatacaatcccttgggtttatttgtttccaagcatgtcattaacttatcttaatgtggacaagaaactgatgcctgcttacattgctattatttcaagcgggtattgatcctttgacatgtgattgatcatttttttttctctggttattagggcacaacagtggtggacaacttgctgaacagtgaggatgttcactacatgcttgaggccctgaaagccctcgggctctctgtggaagcagataaagttgcaaaaagagctgtagtcgttggctgtggtggcaagtttcctgttgagaaggatgcgaaagaggaagtgcaactcttcttgggaaacgctggaattgcaatgcgatcgttgacagcagccgtgactgctgctggtggaaatgcaacgtatgtttttttttaatgtttatgaaaataagtatggaattcatggggtatgttttatgacctttttctttaccatcagttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttagttactcctaaactgcatcctttgtacttctgtatgcacctcaattctttgtcaaccttctgcatttataaggaacattctatgatgaaattcgaccttacactgcacagtaacttgaaatgtttcatgcttaatcaatatgccatattcctgccaagctcaagcgagcaatatttgtttgaatttggtaccatatttttgtatacctgcaggggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 48 - DNA, 9186 bp, Artificial Sequence
  Plasmid KVA790 encoding a modified Cas9
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgggcccggccggccgcgatcgcgcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagccatggcccctaagaagaaacgaaaggtaggcatccacggtgttccagcggctatggacaagaagtacagcatcgggctggacattggcacgaactctgttgggtgggcggttatcactgacgagtacaaggtgccgtcgaagaagttcaaggtgctaggcaacaccgaccgtcacagcatcaagaagaacctcatcggcgccctgttgtttgactctggggaaaccgccgaggctactcgactaaagagaaccgcccgtaggcgatacactaggcggaagaacaggatctgttatctccaggagatcttcagcaacgagatggcgaaggtcgatgactcattcttccaccgccttgaggagtctttcctggtcgaggaggacaaaaagcacgagcgacacccgatcttcgggaacatcgttgatgaggtagcctaccacgagaagtaccctactatctaccacctgcgcaagaaactagtggactccaccgacaaggcggacctccgccttatctatctcgccctcgcccacatgatcaagtttcgcgggcacttcctcatcgaaggcgatctcaatccggacaacagcgacgtggacaagctattcatccagctagttcagacctacaaccagctctttgaagagaaccctattaacgcgagcggggtggacgccaaagccatcctttccgccaggctctctaagagccggagactcgagaaccttatcgctcagttgcccggcgagaagaaaaacggccttttcggcaacctgattgccctgtccttgggcttgacgccgaacttcaagagcaacttcgacctagctgaggatgctaaactgcagctgtccaaggacacttatgatgacgaccttgacaacctcctggcccaaattggggatcagtacgcggacctcttcctcgctgccaagaacctctccgacgcgattctgttgtcggacatcctccgcgtgaacaccgagattactaaggccccactgtccgcgagcatgatcaaacgctacgacgagcaccatcaggatctaactctgctcaaggcgctcgtccgtcagcaactccctgagaagtacaaggagatcttcttcgaccagtccaagaacggctacgccggttacattgatggtggcgcgagccaggaggagttctacaagttcatcaagcccatcttagagaagatggacggcaccgaagagcttctcgtgaagctcaacagggaggacctcctaaggaagcagcgcacctttgacaacgggtctattcctcatcagatccatctcggcgagctccacgcgattttgagacgccaagaggacttctacccgttcttgaaggacaaccgggagaagatcgagaagatcctcacgttcaggatcccttattacgtcggccccctagctaggggtaactcgagattcgcgtggatgaccagaaagtccgaggaaaccattaccccgtggaactttgaagaggtggtcgacaagggcgcgagcgcgcaatcgttcattgagcggatgaccaacttcgacaagaacctaccgaacgagaaggtcctgccgaaacactccctgctctacgagtacttcaccgtttataacgagctgacgaaggtcaagtacgtgactgagggcatgcgtaaaccagcgttcctcagcggtgagcagaagaaggctatcgtggacctgctctttaagacgaaccgcaaggtgaccgtgaagcagctcaaggaggattatttcaagaagatcgagtgcttcgactcagtggagattagcggcgtggaggaccgtttcaatgcgagcttaggtacctaccacgacctcctgaagatcatcaaggacaaggacttcctcgacaacgaggaaaacgaggacatcctggaggacatcgtgctcaccctgaccctctttgaagacagggagatgatcgaggaaaggttgaagacctacgcgcacctattcgacgacaaggtcatgaaacagctcaagcggaggagatacactggctggggcagactttcgcggaagctcatcaatgggatcagggacaagcagtccggcaagaccatcctggacttcttgaagagcgacggcttcgctaatcgcaacttcatgcagctcattcatgacgactccctaacattcaaggaggacatccaaaaggcgcaagtctcgggtcagggggattctttgcacgagcacatcgcaaacctcgccggttctccggccattaaaaagggcatccttcagacggtcaaggtggtcgacgagctcgtgaaggttatgggccggcataagcccgagaacatagtgatcgagatggctagggagaaccagaccacgcaaaagggccagaagaactcacgtgagcgcatgaagaggatcgaggaaggtatcaaggagctgggctctcagattctcaaggagcatccggtcgagaacacccagcttcagaacgagaagctgtacctctactacctccagaatggccgcgacatgtacgttgatcaggagttagatattaatcggctgagcgactacgacgttgaccacatcgtgccccagtcgttcctcaaggatgactccatcgacaacaaggtcctgaccagatcggacaagaaccgtggtaagagcgacaatgtgccctccgaggaggtggtcaaaaagatgaagaactattggcggcagttgctgaacgccaagctcatcacccagcgaaagttcgataatttaacgaaggccgaacgagggggcctttccgaactcgataaggccggtttcatcaagcgtcagctggttgagacgcgacagatcacgaagcatgtggcccagattctggacagccggatgaacacgaagtacgacgagaacgacaaacttattcgggaggtgaaggtgataaccctcaagtccaagttagtgtcggacttccgcaaggactttcagttctataaagtgcgcgagatcaacaattaccaccacgcccacgacgcctacttgaatgccgtggtcggcacggcgctcatcaaaaagtaccccaagctggagagcgagttcgtctacggcgactacaaggtttatgacgtcaggaagatgattgcgaagtcagagcaagagatcgggaaggccaccgccaagtacttcttctactcgaacatcatgaacttctttaagaccgagatcaccctcgccaatggggagatccggaaacgtccccttatcgagacgaacggcgagactggcgagatagtctgggacaaaggtcgcgatttcgccaccgttcgaaaggtgctctcaatgcctcaggtcaatatcgtgaagaagaccgaggttcagacggggggcttcagcaaggagtccatcctccctaagcgcaactctgacaagctgatcgcccggaaaaaggattgggacccgaaaaagtacggcggcttcgattctcccaccgtagcttatagcgtgctcgtggttgccaaggtggagaagggcaagagcaagaaactgaagtcggtcaaggagttactcggcatcacaatcatggagcgcagctcgtttgaaaagaacccgatcgacttcctggaggccaagggctataaggaggtgaagaaggacctcatcattaagctgccgaagtacagcttgttcgagctcgagaatggcaggaagaggatgctagcttcggccggcgaacttcaaaaagggaacgagctcgcgttgccgtcgaagtacgtgaacttcctctacttagctagtcactatgaaaagctgaagggctcgccggaggacaacgagcagaagcagctcttcgtcgagcaacacaagcactacctcgacgagatcatcgagcagataagtgagttcagcaagcgcgtgatcctcgctgatgccaacctcgataaggtgctcagcgcctacaataagcaccgagacaagccgattcgggagcaggcggagaacatcattcatttatttacgctcactaacctaggcgctccggctgccttcaagtacttcgacaccacgatcgacaggaagcgctacaccagcaccaaagaagtcctcgacgcgacgctcattcaccagagcatcacagggctctacgagactcggatcgacttgtcgcaattgggtggggataagaggccggccgcgactaaaaaggccggccaagccaagaaaaagaaatgattgctagcacgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggcccgggttaattaaatttaaatggcgcgccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 49 - protein, 1401 aa, Artificial Sequence [sequence omitted]

SEQ ID NO: 50 - DNA, 23 bp, Triticum aestivum
  misc_feature: Target site TS1
ctaggtgtggagaacatacatgg

SEQ ID NO: 51 - DNA, 23 bp, Triticum aestivum
  misc_feature: Target site TS2
gaaggaggatgggctaggtgtgg

SEQ ID NO: 52 - DNA, 23 bp, Triticum aestivum
  misc_feature: Target site TS3
ataggccctagaataggcactgg

SEQ ID NO: 53 - DNA, 23 bp, Triticum aestivum
  misc_feature: Target site TS4
ctcctcataggccctagaatagg

SEQ ID NO: 54 - DNA, 23 bp, Triticum aestivum
  misc_feature: Target site TS5
ctattgccagtgcctattctagg

SEQ ID NO: 55 - DNA, 23 bp, Triticum aestivum
  misc_feature: Target site TS6
ttcctcgtgctgggcaagtctgg

SEQ ID NO: 56 - DNA, 23 bp, Triticum aestivum
  misc_feature: Target site TS7
gaacaaccagcatctgggaatgg

SEQ ID NO: 57 - DNA, 23 bp, Triticum aestivum
  misc_feature: Target site TS8
atctgggaatggtggtgcagtgg

SEQ ID NO: 58 - DNA, 23 bp, Triticum aestivum
  misc_feature: Target site TS9
ccattgacagcagccgtgactgc

SEQ ID NO: 59 - DNA, 24 bp, Triticum aestivum
  misc_feature: Target site TS10
gaggaagtgcaactcttcttgggg

SEQ ID NO: 60 - DNA, 3645 bp, Artificial Sequence
  Plasmid KVA761 comprising donor DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcgatcgcttctcccaaaattatgcaattttgaggctcctctacatcattataattccccaatacattgctctttattcttaacagctttgatcgcgaaatttaacattttaattcttgagctgttattttgtagcatcagtttatcatgagccatgtttggtactaaatatacaatcccttgggtttatttgtttccaagcatgtcattaacttatcttaatgtggacaagaaactgatgcctgcttacattgctattatttcaagcgggtattgatcctttgacatgtgattgatcatttttttttctctggttattagggcacaacagtggtggacaacttgctgaacagtgaggatgttcactacatgcttgaggccctgaaagccctcgggctctctgtggaagcagataaagttgcaaaaagagctgtagtcgttggctgtggtggcaagtttcctgttgagaaggatgcgaaagaggaagtgcaactcttcttggggaacgctggaattgcaatgcgatcgttgacagcagccgtgactgctgctggtggaaatgcaacgtatgtttttttttaatgtttatgaaaataagtatggaattcatggggtatgttttatgacctttttctttaccatcagttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttagttactcctaaactgcatcctttgtacttctgtatgcacctcaattctttgtcaaccttctgcatttataaggaacattctatgatgaaattcgaccttacactgcacagtaacttgaaatgtttcatgcttaatcaatatgccatattcctgccaagctcaagcgagcaatatttgtttgaatttggtaccatatttttgtatacctgcaggggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2023/0042273 A1
(43) Pub. Date: Feb. 9, 2023

(54) IMPROVED GENOME EDITING USING PAIRED NICKASES

(71) Applicant: BASF Agricultural Solutions Seed US LLC, Florham Park, NJ (US)

(72) Inventors: Katelijn D'HALLUIN, Ghent (BE); Timothy James GOLDS, Ghent (BE); David DE VLEESSCHAUWER, Ghent (BE)

(21) Appl. No.: 17/785,343

(22) PCT Filed: Dec. 7, 2020
(86) PCT No.: PCT/EP2020/084799

§ 371 Date: Jun. 14, 2022

(30) Foreign Application Priority Data
Dec. 16, 2019 (EP) 19216386.3
Feb. 3, 2020 (EP) 20155128.0
Dec. 2, 2020 (EP) 20211151.4

(51) Int. Cl. C12N 15/82

(52) U.S. Cl. CPC C12N 15/8213; C12N 2330/50

(57) ABSTRACT

Genome editing including the introducing of precise gene edits is well established in diploid plants. Methods well established in the art introduce double strand DNA breaks in the genome of a plant applying technologies such as Zn-finger nucleases, homing endonucleases, TALEN or RNA guided nuclease e.g. Cas9 or Cas12a.

### DESCRIPTION OF THE INVENTION

The present invention is in the field of plant molecular biology and is directed to a method for improved genome editing in crops, preferably alloploid and/or polyploid crops.

### INTRODUCTION

Genome editing including the introducing of precise gene edits is well established in diploid plants. Methods well established in the art introduce double strand DNA breaks in the genome of a plant applying technologies such as Zn-finger nucleases, homing endonucleases, TALEN or RNA guided nuclease e.g. Cas9 or Cas12a.

Genome editing applied in plant cells, e.g. embryos, callus or protoplast, is reasonably efficient leading to mutations comprising random insertions and/or deletions (InDels), if the double strand break in the genome is repaired by the error prone non homologous end joining (NHEJ), to unaltered genomic sequences, if the editing approach failed, or to a precise edit (PE), if the break is repaired by homologous recombination, usually the mechanism that occurs the least in plant double strand break repair.

In diploid plants this would lead to the following genotypes: WT/WT, wT/InDel, InDel/InDel, PE/WT, PE/InDel or PE/PE. In cases where precise edits are intended, and random mutations should be avoided the preferred combination would be PE/WT or PE/PE. Screening systems for these combinations are readily available and with the improved efficiencies of genome editing only a reasonable number of cells need to be screened in diploid plants. However, in alloploid and/or polyploid organisms the number of potential combinations increase, and huge numbers of cells need to be screened to avoid plant cells comprising InDel mutations and to identify the preferred combination in more than one genome present in alloploid and/or polyploid plants. In order to reduce the cost- and labor-intensive screening there is a need in the art for methods with reduced percentage of InDels and higher percentage of PE.

Such methods are especially interesting for alloploid and/or polyploid crops, such as wheat, triticale, cotton, potato, oil seed rape, leek, tobacco, peanut, oat, kiwi, banana, strawberry, sugar cane, oca and some apple and kinnow varieties.

NHEJ occurs mostly in cases in which no DNA allowing for HR repair is present at the double strand DNA break. HR repair requires DNA regions having certain degree of homology to the DNA at or in close vicinity to the double strand break. This homologous DNA may be present within the genome of the plant or may be present on donor DNA comprising at the 3′ and/or 5′ end regions with a certain degree of homology to the genomic DNA at or in close vicinity to the double strand break. However, even if a donor DNA is introduced into a cell together with the double strand break inducing agent, it may not be present at the break site at the time, the DNA repair occurs.

The present invention provides a method using paired nickases nicking one or both strands of double stranded DNA without leading to physical separation of the double stranded DNA. Such nicks would not lead to a double strand break but the base pairs between the nicks would keep the complementary DNA strands together by keeping the hydrogen bonds between the complementary bases of the two strands intact. A repair would either lead to WT sequence or a precise gene edit, in case a respective donor DNA molecule with homologous overhangs at the 3′ and/or 5′ end is present at the nick at the time of repair and such the percentage of random InDel mutations is reduced.

EP3138912 discloses paired Cas9 nickases to introduce a double strand break into the genome of a plant cell to reduce the percentage of off-target double strand breaks introduced by a single Cas9 nuclease binding at non target sites having a certain homology to the guide RNA. The authors explicitly point out, that the nickases need to nick in close enough proximity to induce double strand breaks. However, they give no guidance what distance would be close enough to introduce double strand breaks and they are silent about the problem of reducing the percentage of InDels in the repair process.

Mali et al (2013) disclose the use of paired Cas9 nickases in diploid human cells to induce InDels without codelivery of donor DNA molecules.

Schiml et al (2014) and Fauser et al (2014) describe the use of paired Cas9 nickases or a single Cas9 nickase in diploid Arabidopsis cells to induce intrachromosomal homologous recombination without codelivery of donor DNA molecules.

Mikami et al (2016) describe the use of paired Cas9 nickases in diploid rice cells to reduce the percentage of off-target mutations without codelivery of donor DNA molecules.

Wolter et al (2018) disclose the use of paired Cas9 nickases in diploid Arabidopsis cells to induce intrachromosomal homologous recombination without codelivery of donor DNA molecules. They further show in an in planta gene targeting system in Arabidopsis, that relies on a donor DNA excised from the plants genome prior to recombination at a different locus of the genome, that only introduction of double-strand DNA breaks at the target locus lead to a significant number of precise gene edits in the plants genome, whereas no or hardly any true gene targeting events were identified using a nickase or paired nickases. The major fraction of the events obtained with a paired nickase were ectopic recombination events. There is a need in the art for the efficient and reliable introduction of donor DNA into predefined areas of the genome of alloploid and/or polyploid plants, preferably alloploid and/or polyploid crops, using the recently developed CRISPR method. Moreover, there is a need in the art for increasing efficiency of introduction of donor DNA into the genome of plants, preferably alloploid and/or polyploid plants, e.g. alloploid and/or polyploid crops, by reducing the proportion of InDels occurring in the plant genome.

### DETAILED DESCRIPTION OF THE INVENTION

A first embodiment of the invention comprises a method for introducing at least one donor DNA molecule into at least one target region of the genome of a plant cell, preferably a crop plant cell, more preferably an alloploid or polyploid or alloploid and polyploid crop plant cell, most preferably a wheat cell comprising the steps of
a. Introducing into said plant cell
   i. a donorDNA molecule and
   ii. at least one RNA guided nickase and
   iii. at least two single guide RNAs (sgRNAs) or at least two CRISPR RNA (crRNA) and trans-activating RNA (tracrRNA) and
b. Incubating the plant cell to allow for introduction of said at least one donor DNA into said at least one target region of the genome, and
c. Selecting a plant cell comprising the sequence of the donor DNA molecule in said target region,

wherein the nickase creates at least two nicks on opposite strands or on one strand at the target site, i. e. in or near the target region of the genomic DNA of the plant cell, preferably a crop plant cell, more preferably an alloploid or polyploid or alloploid and polyploid crop plant cell, most preferably a wheat cell and

wherein these nicks are at least 20 base pairs apart from each other and

wherein the base pairs between the nicks are not dissolved and keep the DNA double strand together by keeping the hydrogen bonds between the complementary bases intact, and

wherein each nicking site is adjacent to at least one PAM sequence and wherein the at least two sgRNA or the at least two tracrRNA and crRNA are targeting the at least one RNA guided nickase to the target sites.

In a preferred embodiment the nicks are at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145 or 150 base pairs apart from each other but not more than 200, 195, 190, 185, 180, 175, 170, 165, 160 or 155 base pairs apart from each other.

In one embodiment the donor DNA is functionally linked to at least 30 bases at its 5′ and/or 3′ end that are each at least 80% identical to a sequence in the target region, preferably the donor DNA is functionally linked at its 5′ and 3′ end to such sequence. Preferably the sequence at at least one side of the donor DNA, preferably at both sides of the donor DNA comprises at least 40, at least 50, at least 60, at least 70, at least 80, at least 90 or at least 100 bases. More preferably the sequence at at least one side of the donor DNA, preferably at both sides of the donor DNA comprises at least 150 bases, at least 200 bases, at least 300 bases, at least 350 bases or at least 400 bases. These bases are at least 80%, preferably at least 85%, preferably 90%, preferably 91%, 92%, 93% or 94% identical to the respective 5′ and 3′ region of the double strand or single strand nick introduced by the RNA guided nickase. More preferably these bases are at least 95% identical, 96% identical, 97% identical, 98% identical or 99% identical to the respective 5′ and 3′ region of the double strand or single strand nick introduced by the RNA guided nickase. In a most preferred embodiment, these bases are 100% identical to the respective 5′ and 3′ region of the double strand or single strand nick introduced by the RNA guided nickase.

In one embodiment, the at least 30 bases at the 5′ and/or 3′ end of the donor DNA are 100% identical to the respective 5′ and/or 3′ region of the double strand or single strand nick where the donor DNA or its sequence are inserted in the genomic DNA. In another embodiment the at least 40 or 50 bases at the 5′ and/or 3′ end of the donor DNA are at least 98% identical to the respective 5′ and/or 3′ region of the double strand or single strand nick. In a further embodiment the at least 60 or 70 bases at the 5′ and/or 3′ end of the donor DNA are at least 95% identical to the respective 5′ and/or 3′ region of the double strand or single strand nick. In a preferred embodiment the at least 80 or 90 bases at the 5′ and/or 3′ end of the donor DNA are at least 92% identical to the respective 5′ and/or 3′ region of the double strand or single strand nick. In a more preferred embodiment, the at least 100 bases at the 5′ and/or 3′ end of the donor DNA are at least 90% identical to the respective 5′ and/or 3′ region of the double strand or single strand nick. In a more preferred embodiment, the at least 150 or 200 bases at the 5′ and/or 3′ end of the donor DNA are at least 85% identical to the respective 5′ and/or 3′ region of the double strand or single strand nick. In a further preferred embodiment, the at least 250, 300, 350 or 400 at the 5′ and/or 3′ end of the donor DNA are at least 80% identical to the respective 5′ and/or 3′ region of the double strand or single strand nick.

In one embodiment of the invention the donor DNA molecule is single stranded, in another embodiment, the donor DNA molecule is double stranded. In one embodiment the donor DNA molecule is not more than 10 nucleotides in length, in another embodiment it is not more than 20, 30 40 or 50 nucleotides in length. In another embodiment the donor DNA molecule is not more than 60, 70, 80, 90 or 100 nucleotides in length. In another embodiment, the donor DNA molecule is not more than 125, 150, 200, 300, 400 or 500 nucleotides in length. In another embodiment, the donor DNA molecule is not more than 600, 700, 800, 900, 1000, 1100, 1200, 1300, 1400 or 1500 nucleotides in length. In another embodiment, the donor DNA molecule is not more than 2000, 2500, 3000, 3500, 4000, 4500 or 5000 nucleotides in length.

In one embodiment the donor DNA molecule is added to the target region of the genome of the alloploid or polyploid plant, preferably alloploid or polyploid crop and does not replace genomic DNA. In another embodiment the donor DNA molecule replaces a sequence in the target region of the alloploid or polyploid plant, preferably alloploid or polyploid crop genome which is shorter, the same size or longer than the donor DNA molecule.

In one embodiment the donor DNA molecule comprises sequences not present at the target region of the alloploid or polyploid plant, preferably alloploid or polyploid crop genome. By introduction of such DNA molecules in the target region of the alloploid or polyploid plant, preferably alloploid or polyploid crop genome additional DNA is added to the genome that may comprise regulatory regions such as a promoter, an intron, enhancer or terminator, it may comprise transcribed regions such as ORFs or may encode non coding RNAs such as microRNA precursors, long noncoding RNAs and the like or it may comprise one or more expression constructs. In another embodiment the donor DNA molecule comprises sequences homologous to the target region of the alloploid or polyploid plant, preferably alloploid or polyploid crop genome but is comprising one or more precise gene edits that differ from the WT sequence at the target region of the genome. Such donor DNA molecules are replacing corresponding sequences in the genome thereby introducing precise gene edits into the alloploid or polyploid plant, preferably alloploid or polyploid crop genome. The plant cell is preferably derived from an alloploid or polyploid plant such as chrysanthemum, dahlia or saffron crocus, preferably an alloploid or polyploid crop, for example wheat, triticale, cotton, potato, oil seed rape, leek, tobacco, peanut, oat, kiwi, banana, strawberry, seedless water melon, banana, citrus, sugar cane, oca and some apple and kinnow varieties.

Incubation of the plant cell to allow for introduction of the donor DNA into the genome of the cell may occur at any condition favourable for maintaining the viability of the cell. Temperature is preferably between 20° C. and 32° C., depending for example on the RNA guided nickase used. With respect to Cas9 nickase (nCas9), the temperature is preferably between 18° C. and 30° C., more preferably between 20° C. and 28° C., most preferably between 22° C. and 26° C. With respect to Cas12a nickase (nCas12a), the temperature is preferably between 22° C. and 32° C., more preferably between 24° C. and 30° C., most preferably between 28° C. and 30° C.

The cells are preferably incubated under 16 h light/8 h dark conditions, preferably under dim light conditions, more preferably in the dark. Incubation time is between 1 day and 7 weeks under said conditions, preferably between 5 weeks and 7 weeks.

The RNA guided nickase is guided to the target site by the annealed crRNA and tracrRNA or the single guide RNA respectively. The target site is adjacent to a PAM sequence which is specific for the RNA guided nickase used.

If two target sites are nicked in the genomic DNA of the respective cell, at least two annealed crRNA and tracrRNA or at least two single guide RNAs or at least one annealed crRNA and tracrRNA and at least one single guide RNA are introduced into the cell, each targeting the respective nickase to its target site adjacent to a PAM sequence.

A further embodiment of the invention is a method for producing a plant preferably a crop plant, more preferably an alloploid or polyploid crop plant, most preferably a wheat plant comprising a donor DNA, the donor DNA preferably comprising a precise gene edit, comprising the steps of
a. Introducing into a cell of said plant
   i. a donorDNA molecule and
   ii. at least one RNA guided nickase and
   iii. at least two sgRNAs or at least two crRNA and tracrRNA and
b. Incubating the plant cell to allow for introducing said at least one donor DNA into the target region of the genome of said plant cell, and
c. Selecting a plant cell comprising the sequence of the donor DNA molecule in said target region, and
d. Regenerating a plant from said selected plant cell,

wherein the nickase creates at least two nicks on opposite strands or on one strand at the target site, i. e. in or near the target region of the genomic DNA of the plant cell, preferably a crop plant cell, more preferably an alloploid or polyploid crop plant cell, most preferably a wheat cell and wherein these nicks are at least 20 bases apart from each other and

wherein the base pairs between the nicks are not dissolved and keep the DNA double strand together by keeping the hydrogen bonds between the complementary bases intact, and

wherein each nicking site is adjacent to at least one PAM sequence

and wherein the at least two sgRNA or the at least two tracrRNA and crRNA are targeting the at least one RNA guided nickase to the target site.

In a preferred embodiment the nicks are at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145 or 150 base pairs apart from each other but not more than 200, 195, 190, 185, 180, 175, 170, 165, 160 or 155 base pairs apart from each other. In one embodiment the donor DNA is functionally linked to at least 30 bases at its 5′ and/or 3′ end that are each at least 80% identical to a sequence in the target region, preferably the donor DNA is functionally linked at its 5′ and 3′ end to such sequence. Preferably the sequence at at least one side of the donor DNA, preferably at both sides of the donor DNA comprises at least 40, at least 50, at least 60, at least 70, at least 80, at least 90 or at least 100 bases. More preferably the sequence at at least one side of the donor DNA, preferably at both sides of the donor DNA comprises at least 150 bases, at least 200 bases, at least 300 bases, at least 350 bases or at least 400 bases. These bases are at least 80%, preferably at least 85%, preferably 90%, preferably 91%, 92%, 93% or 94% identical to the respective 5′ and 3′ region of the double strand or single strand nick introduced by the RNA guided nickase. More preferably these bases are at least 95% identical, 96% identical, 97% identical, 98% identical or 99% identical to the respective 5′ and 3′ region of the double strand or single strand nick introduced by the RNA guided nickase. In a most preferred embodiment, these bases are 100% identical to the respective 5′ and 3′ region of the double strand or single strand nick introduced by the RNA guided nickase.

In one embodiment, the at least 30 bases at the 5′ and/or 3′ end of the donor DNA are 100% identical to the respective 5′ and/or 3′ region of the double strand or single strand nick where the donor DNA or its sequence are inserted in the genomic DNA. In another embodiment the at least 40 or 50 bases at the 5′ and/or 3′ end of the donor DNA are at least 98% identical to the respective 5′ and/or 3′ region of the double strand or single strand nick. In a further embodiment the at least 60 or 70 bases at the 5′ and/or 3′ end of the donor DNA are at least 95% identical to the respective 5′ and/or 3′ region of the double strand or single strand nick. In a preferred embodiment the at least 80 or 90 bases at the 5′ and/or 3′ end of the donor DNA are at least 92% identical to the respective 5′ and/or 3′ region of the double strand or single strand nick. In a more preferred embodiment, the at least 100 bases at the 5′ and/or 3′ end of the donor DNA are at least 90% identical to the respective 5′ and/or 3′ region of the double strand or single strand nick. In a more preferred embodiment, the at least 150 or 200 bases at the 5′ and/or 3′ end of the donor DNA are at least 85% identical to the respective 5′ and/or 3′ region of the double strand or single strand nick. In a further preferred embodiment, the at least 250, 300, 350 or 400 at the 5′ and/or 3′ end of the donor DNA are at least 80% identical to the respective 5′ and/or 3′ region of the double strand or single strand nick.

In one embodiment of the invention the donor DNA molecule is single stranded, in another embodiment, the donor DNA molecule is double stranded. In one embodiment the donor DNA molecule is not more than 10 nucleotides in length, in another embodiment it is not more than 20, 30 40 or 50 nucleotides in length. In another embodiment the donor DNA molecule is not more than 60, 70, 80, 90 or 100 nucleotides in length. In another embodiment, the donor DNA molecule is not more than 125, 150, 200, 300, 400 or 500 nucleotides in length. In another embodiment, the donor DNA molecule is not more than 600, 700, 800, 900, 1000, 1100, 1200, 1300, 1400 or 1500 nucleotides in length. In another embodiment, the donor DNA molecule is not more than 2000, 2500, 3000, 3500, 4000, 4500 or 5000 nucleotides in length.

In one embodiment the donor DNA molecule is added to the target region of the genome of the alloploid or polyploid plant, preferably alloploid or polyploid crop and does not replace genomic DNA. In another embodiment the donor DNA molecule replaces a sequence in the target region of the alloploid or polyploid plant, preferably alloploid or polyploid crop genome which is shorter, the same size or longer than the donor DNA molecule.

In one embodiment the donor DNA molecule comprises sequences not present at the target region of the alloploid or polyploid plant, preferably alloploid or polyploid crop genome. By introduction of such DNA molecules in the target region of the alloploid or polyploid plant, preferably alloploid or polyploid crop genome additional DNA is added to the genome that may comprise regulatory regions such as a promoter, an intron, enhancer or terminator, it may comprise transcribed regions such as ORFs or may encode non coding RNAs such as microRNA precursors, long noncoding RNAs and the like or it may comprise one or more expression constructs. In another embodiment the donor DNA molecule comprises sequences homologous to the target region of the alloploid or polyploid plant, preferably alloploid or polyploid crop genome but is comprising one or more precise gene edits that differ from the WT sequence at the target region of the genome. Such donor DNA molecules are replacing corresponding sequences in the genome thereby introducing precise gene edits into the alloploid or polyploid plant, preferably alloploid or polyploid crop genome.

The plant cell is preferably derived from an alloploid or polyploid plant such as chrysanthemum, dahlia or saffron crocus, preferably an alloploid or polyploid crop, for example wheat, triticale, cotton, potato, oil seed rape, leek, tobacco, peanut, oat, kiwi, banana, strawberry, seedless water melon, banana, citrus, sugar cane, oca and some apple and kinnow vaneties.

Incubation of the plant cell to allow for introduction of the donor DNA into the genome of the cell may occur at any condition favourable for maintaining the viability of the cell. Temperature is preferably between 20° C. and 32° C., depending for example on the RNA guided nickase used. With respect to Cas9 nickase (nCas9), the temperature is preferably between 18° C. and 30° C., more preferably between 20° C. and 28° C., most preferably between 22° C. and 26° C. With respect to Cas12a nickase (nCas12a), the temperature is preferably between 22° C. and 32° C., more preferably between 24° C. and 30° C., most preferably between 28° C. and 30° C.

The cells are preferably incubated under 16 h light/8 h dark conditions, preferably under dim light conditions, more preferably in the dark. Incubation time is between 1 day and 7 weeks under said conditions, preferably between 5 weeks and 7 weeks.

The RNA guided nickase is guided to the target site by the annealed crRNA and tracrRNA or the single guide RNA respectively. The target site is adjacent to a PAM sequence which is specific for the RNA guided nickase used.

If two target sites are nicked in the genomic DNA of the respective cell, at least two annealed crRNA and tracrRNA or at least two single guide RNAs or at least one annealed crRNA and tracrRNA and at least one single guide RNA are introduced into the cell, each targeting the respective nickase to its target site adjacent to a PAM sequence.

A further embodiment of the invention is a method as described above, wherein after step b. the plant cell is incubated on a medium comprising a selection agent.

Negative selection markers confer a resistance to a biocidal compound such as a metabolic inhibitor (e.g., 2-deoxyglucose-6-phosphate, WO 98/45456), antibiotics (e.g., kanamycin, G 418, bleomycin or hygromycin) or herbicides (e.g., phosphinothricin or glyphosate). Especially preferred negative selection markers are those which confer resistance to herbicides.

Some of these markers can be used—beside their function as a marker—to confer a herbicide resistance trait to the resulting plant. Examples, which may be mentioned, are:
Phosphinothricin acetyltransferases (PAT; also named Bialophos resistance; bar; de Block et al. (1987) EMBO J 6:2513-2518; EP 0 333 033; U.S. Pat. No. 4,975,374)
5-enolpyruvylshikimate-3-phosphate synthase (EPSPS; U.S. Pat. No. 5,633,435) or glyphosate oxidoreductase gene (U.S. Pat. No. 5,463,175) conferring resistance to Glyphosate (Nphosphonomethyl glycine) (Shah et al. (1986) Science 233: 478)
Glyphosate degrading enzymes (Glyphosate oxidoreductase; gox),
Dalapon inactivating dehalogenases (deh)
Sulfonylurea- and imidazolinone-inactivating acetolactate synthases (for example mutated ALS variants with, for example, the S4 and/or Hra mutation
Bromoxynil degrading nitrilases (bxn)
Kanamycin- or. G418- resistance genes (NPTII; NPTI) coding e.g., for neomycin phosphotransferases (Fraley et al. (1983) Proc Natl Acad Sci USA 80:4803), which expresses an enzyme conferring resistance to the antibiotic kanamycin and the related antibiotics neomycin, paromomycin, gentamicin, and G418,
2-Deoxyglucose-6-phosphate phosphatase (DOGR1-Gene product; WO 98/45456; EP 0 807 836) conferring resistance against 2-desoxyglucose (Randez-Gil et al. (1995) Yeast 11:1233-1240)
Hygromycin phosphotransferase (HPT), which mediates resistance to hygromycin (Vanden Elzen et al. (1985) Plant Mol Biol. 5:299).
Dihydrofolate reductase (Eichholtz et al. (1987) Somatic Cell and Molecular Genetics 13, 67-76)

Additional negative selectable marker genes of bacterial origin that confer resistance to antibiotics include the aadA gene, which confers resistance to the antibiotic spectinomycin, gentamycin acetyl transferase, streptomycin phosphotransferase (SPT), aminoglycoside-3-adenyl transferase and the bleomycin resistance determinant (Svab et al. (1990) Plant Mol. Biol. 14:197; Jones et al. (1987) Mol. Gen. Genet. 210:86; Hille et al. (1986) Plant Mol. Biol. 7:171 (1986); Hayford et al. (1988) Plant Physiol. 86:1216).

Negative selection markers may further confer resistance against the toxic effects imposed by D-amino acids like e.g., D-alanine and D-serine (WO 03/060133; Erikson et al. (2004) Nat Biotechnol. 22(4):455-8), for example the daol gene (EC: 1.4. 3.3: GenBank Acc.-No.: U60066) from the yeast Rhodotorula gracilis (Rhodosporidium toruloides) and the E. coli gene dsdA (D-serine dehydratase (D-serine deaminase) [EC: 4.3. 1.18; GenBank Acc.-No.: J01603). Depending on the employed D-amino acid the D-amino acid oxidase markers can be employed as dual function marker offering negative selection (e.g., when combined with for example D-alanine or D-serine) or counter selection (e.g., when combined with D-leucine or D-isoleucine).

Alternatively, positive selection markers may be applied in the methods of the invention. Such positive selection markers are conferring a growth advantage to a transformed plant in comparison with a non-transformed one. Genes like isopentenyltransferase from Agrobacterium tumefaciens (strain:P022; Genbank Acc.-No.: AB025109) may—as a key enzyme of the cytokinin biosynthesis—facilitate regeneration of transformed plants (e.g., by selection on cytokinin-free medium). Corresponding selection methods are described (Ebinuma et al. (2000a) Proc Natl Acad Sci USA 94:2117-2121; Ebinuma et al. (2000b) Selection of Marker-free transgenic plants using the oncogenes (ipt, rol A, B, C) of Agrobacterium as selectable markers, In Molecular Biology of Woody Plants. Kluwer Academic Publishers). Additional positive selection markers, which confer a growth advantage to a transformed plant in comparison with a non-transformed one, are described e.g., in EP-A 0 601 092. Growth stimulation selection markers may include (but shall not be limited to) Glucuronidase (in combination with e.g., cytokinin glucuronide), mannose-6-phosphate isomerase (in combination with mannose), UDP-galactose-4-epimerase (in combination with e.g., galactose).

Counter selection markers are especially suitable to select organisms with defined deleted sequences comprising said marker (Koprek et al. (1999) Plant J 19(6): 719-726). Examples for counter selection marker comprise thymidine kinases (TK), cytosine deaminases (Gleave et al. (1999) Plant Mol Biol. 40(2):223-35; Perera et al. (1993) Plant Mol. Biol 23(4): 793-799; Stougaard (1993) Plant J 3:755-761), cytochrom P450 proteins (Koprek et al. (1999) Plant J 19(6): 719-726), haloalkan dehalogenases (Naested (1999) Plant J 18:571-576), iaaH gene products (Sundaresan et al. (1995) Gene Develop 9: 1797-1810), cytosine deaminase codA (Schlaman and Hooykaas (1997) Plant J 11:1377-1385), or tms2 gene products (Fedoroff and Smith (1993) Plant J 3:273-289).

In the methods of the invention the RNA guided nickase may be any RNA guided nickase, preferably they are Cas nickases. The skilled person is aware of many Cas nickases that are described in the art. For example, Cas9, Cas12a, Cas12b, CasX, CasY, C2c1, C2c3, C2c2, Cas12k and the like.

Also, methods for identifying new Cas nickases are described (U.S. Pat. No. 9,790,490) and allow the skilled person to isolate further yet unknown Cas nickases.

In a preferred embodiment of the invention the Cas nickase is a Cas9 or Cas12a nickase or an inactive Cas (dCas) e.g. dCas9 or dCas12a fusion protein fused to a nickase activity, such as, for example Fokl nickase (U.S. Pat. No. 9,200,266).

In a further embodiment of the methods of the invention the nickase or the at least one sgRNA or at least one crRNA and tracrRNA is introduced into said cell encoded by a nucleic acid molecule. Said nucleic acid molecule may be an RNA molecule or a linear DNA molecule encoding the respective nickase, sgRNA, crRNA and/or tracrRNA, preferably the nucleic acid molecule is a plasmid comprising an expression cassette encoding said at least one nickase or the at least one sgRNA or at least one crRNA and tracrRNA.

In a preferred embodiment the at least one nickase is sequence optimized for expression in the respective alloploid or polyploid plant. Sequence optimization is a technology known to the skilled person. Computer programs are available that adapt any given DNA or RNA molecule to the preferred codon usage of the organism in which the respective protein shall be expressed. Some programs additionally allow the mutation of cryptic splice sides, reduction of RNA folding and the like.

The RNA guided nickase and the at least one sgRNA or at least one crRNA and tracrRNA may be introduced into the cell using any method known to a skilled person. Methods like Agrobacterium mediated transformation, transfection using PEG, lipoproteins or other polypeptides, electroporation or ballistic methods such as particle bombardment may be applied. Preferably the at least one RNA guided nickase and the at least one sgRNA or at least one crRNA and tracrRNA are introduced into said cell as ribonucleoprotein (RNP) assembled outside said cell.

In a preferred embodiment of the methods of the invention a combination of donorDNA and crRNA/tracrRNA or sgRNA is preselected for efficient introduction of the donor DNA molecute into the target region. In a preferred embodiment of the methods of the invention the at least one donor DNA and at least one RNA guided nickase and at least one singleguideRNA (sgRNA) or tracrRNA and crRNA are introduced into said cell using particle bombardment or Agrobacterium mediated introduction of DNA.

Preferably the at least one RNA guided nickase is comprising a nuclear localization signal.

### Definitions

Abbreviations: GFP—green fluorescence protein, GUS—beta-Glucuronidase, BAP—6-benzylaminopurine; 2,4-D—2,4-dichlorophenoxyacetic acid; MS—Murashige and Skoog medium; NAA—1-naphtaleneacetic acid; MES, 2-(N-morpholino-ethanesulfonic acid, IAA indole acetic acid; Kan: Kanamycin sulfate; GA3—Gibberellic acid; Timentin™: ticarcillin disodium/clavulanate potassium, microl: Microliter.

It is to be understood that this invention is not limited to the particular methodology or protocols. It is also to be understood that the terminology used herein is for the purpose of describing particular embodiments only and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,”“comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Antisense: The term “antisense” refers to a nucleotide sequence that is inverted relative to its normal orientation for transcription or function and so expresses an RNA transcript that is complementary to a target gene mRNA molecule expressed within the host cell (e.g., it can hybridize to the target gene mRNA molecule or single stranded genomic DNA through Watson-Crick base pairing) or that is complementary to a target DNA molecule such as, for example genomic DNA present in the host cell.

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, posttranscriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

donor DNA molecule: As used herein the terms “donor DNA molecule”, “repair DNA molecule” or “template DNA molecule” all used interchangeably herein mean a DNA molecule having a sequence that is to be introduced into the genome of a cell. It may be flanked at the 5′ and/or 3′ end by sequences homologous or identical to sequences in the target region of the genome of said cell. It may comprise sequences not naturally occurring in the respective cell such as ORFs, non-coding RNAs or regulatory elements that shall be introduced into the target region or it may comprise sequences that are homologous to the target region except for at least one mutation, a gene edit: The sequence of the donor DNA molecule may be added to the genome or it may replace a sequence in the genome of the length of the donor DNA sequence.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of the untransformed plant cell.

Enhanced expression: “enhance” or “increase” the expression of a nucleic acid molecule in a plant cell are used equivalently herein and mean that the level of expression of the nucleic acid molecule in a plant, part of a plant or plant cell after applying a method of the present invention is higher than its expression in the plant, part of the plant or plant cell before applying the method, or compared to a reference plant lacking a recombinant nucleic acid molecule of the invention. For example, the reference plant is comprising the same construct which is only lacking the respective NEENA. The term “enhanced” or “increased” as used herein are synonymous and means herein higher, preferably significantly higher expression of the nucleic acid molecule to be expressed. As used herein, an “enhancement” or “increase” of the level of an agent such as a protein, mRNA or RNA means that the level is increased relative to a substantially identical plant, part of a plant or plant cell grown under substantially identical conditions, lacking a recombinant nucleic acid molecule of the invention, for example lacking the NEENA molecule, the recombinant construct or recombinant vector of the invention. As used herein, “enhancement” or “increase” of the level of an agent, such as for example a preRNA, mRNA, rRNA, tRNA, snoRNA, snRNA expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a cell or organism lacking a recombinant nucleic acid molecule of the invention. The enhancement or increase can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254). As one example for quantifying the activity of a protein, the detection of luciferase activity is described in the Examples below.

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein mean a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, ta-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus. In the case of a plant, the promoter can also be specific to a particular tissue or organ or stage of development.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” is to be understood as meaning, for example, the sequential arrangement of a regulatory element (e.g. a promoter) with a nucleic acid sequence to be expressed and, if appropriate, further regulatory elements (such as e.g., a terminator or a NEENA) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (N.Y.); Silhavy et al. (1984) Experiments with Gene Fusions, Cold Spring Harbor Laboratory, Cold Spring Harbor (N.Y.); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

“Gene edit” when used herein means the introduction of a specific mutation at a specific position of the genome of a cell. The gene edit may be introduced by precise editing applying more advanced technologies e.g. using a CRISPR Cas system and a donor DNA, or a CRISPR Cas system linked to mutagenic activity such as a deaminase (WO15133554, WO17070632).

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Heterologous: The term “heterologous” with respect to a nucleic acid molecule or DNA refers to a nucleic acid molecule which is operably linked to, or is manipulated to become operably linked to, a second nucleic acid molecule, e.g. a promoter to which it is not operably linked in nature, e.g. in the genome of a WT plant, or to which it is operably linked at a different location or position in nature, e.g. in the genome of a WT plant.

Preferably the term “heterologous” with respect to a nucleic acid molecule or DNA, e.g. a NEENA refers to a nucleic acid molecule which is operably linked to, or is manipulated to become operably linked to, a second nucleic acid molecule, e.g. a promoter to which it is not operably linked in nature.

A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

High expression promoter: A “high expression promoter” as used herein means a promoter causing expression in a plant or part thereof wherein the accumulation or rate of synthesis of RNA or stability of RNA derived from the nucleic acid molecule under the control of the respective promoter is higher, preferably significantly higher than the expression caused by the promoter lacking the NEENA of the invention. Preferably the amount of RNA and/or the rate of RNA synthesis and/or stability of RNA is increased 50% or more, for example 100% or more, preferably 200% or more, more preferably 5-fold or more, even more preferably 10-fold or more, most preferably 20-fold or more for example 50-fold relative to a promoter lacking a NEENA of the invention.

Hybridization: The term “hybridization” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specifis sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules. The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

DNA-DNA hybrids (Meinkoth and Wahl, Anal. Biochem., 138: 267-284, 1984):

Tm=81.5° C.+16.6×log[Na+]a+0.41×%[G/Cb]−500×[Lc]−1−0.61×% formamide

DNA-RNA or RNA-RNA hybrids:

Tm=79.8+18.5(log 10[Na+]a)+0.58 (% G/Cb)+11.8 (% G/Cb)2−820/Lc

oligo-DNA or oligo-RNAd hybrids:

For <20 nucleotides: Tm=2 (ln)

For 20-35 nucleotides: Tm=22+1.46 (ln)

a or for other monovalent cation, but only accurate in the 0.01-0.4 M range.

b only accurate for % GC in the 30% to 75% range.

c L=length of duplex in base pairs.

d Oligo, oligonucleotide; In, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

“Identity”: “Identity” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases

Seq B: GATCTGA length: 7 bases

Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “|” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %−identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

InDel is a term for the random insertion or deletion of bases in the genome of an organism associated with the repair of a DSB by NHEJ. It is classified among small genetic variations, measuring from 1 to 10 000 base pairs in length. As used herein it refers to random insertion or deletion of bases in or in the close vicinity (e.g. less than 1000 bp, 900 bp, 800 bp, 700 bp, 600 bp, 500 bp, 400 bp, 300 bp, 250 bp, 200 bp, 150 bp, 100 bp, 50 bp, 40 bp, 30 bp, 25 bp, 20 bp, 15 bp, 10 bp or 5 bp up and/or downstream) of the target site.

The term “Introducing”, “introduction” and the like with respect to the introduction of a donor DNA molecule in the target site of a target DNA means any introduction of the sequence of the donor DNA molecule into the target region for example by the physical integration of the donor DNA molecule or a part thereof into the target region or the introduction of the sequence of the donor DNA molecule or a part thereof into the target region wherein the donor DNA is used as template for a polymerase.

Intron: refers to sections of DNA (intervening sequences) within a gene that do not encode part of the protein that the gene produces, and that is spliced out of the mRNA that is transcribed from the gene before it is exported from the cell nucleus. Intron sequence refers to the nucleic acid sequence of an intron. Thus, introns are those regions of DNA sequences that are transcribed along with the coding sequence (exons) but are removed during the formation of mature mRNA. Introns can be positioned within the actual coding region or in either the 5′ or 3′ untranslated leaders of the pre-mRNA (unspliced mRNA). Introns in the primary transcript are excised and the coding sequences are simultaneously and precisely ligated to form the mature mRNA. The junctions of introns and exons form the splice site. The sequence of an intron begins with GU and ends with AG. Furthermore, in plants, two examples of AU-AC introns have been described: the fourteenth intron of the RecA-like protein gene and the seventh intron of the G5 gene from Arabidopsis thaliana are AT-AC introns. Pre-mRNAs containing introns have three short sequences that are—beside other sequences—essential for the intron to be accurately spliced. These sequences are the 5′ splice-site, the 3′ splice-site, and the branchpoint. mRNA splicing is the removal of intervening sequences (introns) present in primary mRNA transcripts and joining or ligation of exon sequences. This is also known as cis-splicing which joins two exons on the same RNA with the removal of the intervening sequence (intron). The functional elements of an intron is comprising sequences that are recognized and bound by the specific protein components of the spliceosome (e.g. splicing consensus sequences at the ends of introns). The interaction of the functional elements with the spliceosome results in the removal of the intron sequence from the premature mRNA and the rejoining of the exon sequences. Introns have three short sequences that are essential—although not sufficient—for the intron to be accurately spliced. These sequences are the 5′ splice site, the 3′ splice site and the branch point. The branchpoint sequence is important in splicing and splice-site selection in plants. The branchpoint sequence is usually located 10-60 nucleotides upstream of the 3′ splice site.

Isogenic: organisms (e.g., plants), which are genetically identical, except that they may differ by the presence or absence of a heterologous DNA sequence.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO:1 where the nucleic acid sequence is in a chromosomal or extrachromosomal location different from that of natural cells or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: promoter elements, particularly a TATA element, that are inactive or that have greatly reduced promoter activity in the absence of upstream activation. In the presence of a suitable transcription factor, the minimal promoter functions to permit transcription.

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acid expression enhancing nucleic acid (NEENA): The term “nucleic acid expression enhancing nucleic acid” refers to a sequence and/or a nucleic acid molecule of a specific sequence having the intrinsic property to enhance expression of a nucleic acid under the control of a promoter to which the NEENA is functionally linked. Unlike promoter sequences, the NEENA as such is not able to drive expression. In order to fulfill the function of enhancing expression of a nucleic acid molecule functionally linked to the NEENA, the NEENA itself has to be functionally linked to a promoter. In distinction to enhancer sequences known in the art, the NEENA is acting in cis but not in trans and has to be located close to the transcription start site of the nucleic acid to be expressed.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or anti-sense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Plant: is generally understood as meaning any eukaryotic single- or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred. The term includes the mature plants, seed, shoots and seedlings and their derived parts, propagation material (such as seeds or microspores), plant organs, tissue, protoplasts, callus and other cultures, for example cell cultures, and any other type of plant cell grouping to give functional or structural units. Mature plants refer to plants at any desired developmental stage beyond that of the seedling. Seedling refers to a young immature plant at an early developmental stage. Annual, biennial, monocotyledonous and dicotyledonous plants are preferred host organisms for the generation of transgenic plants. The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, Hepaticae (liverworts) and Musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae. Preferred are plants which are used for food or feed purpose such as the families of the Leguminosae such as pea, alfalfa and soya; Gramineae such as rice, maize, wheat, barley, sorghum, millet, rye, triticale, or oats; the family of the Umbelliferae, especially the genus Daucus, very especially the species carota (carrot) and Apium, very especially the species Graveolens dulce (celery) and many others; the family of the Solanaceae, especially the genus Lycopersicon, very especially the species esculentum (tomato) and the genus Solanum, very especially the species tuberosum (potato) and melongena (egg plant), and many others (such as tobacco); and the genus Capsicum, very especially the species annuum (peppers) and many others; the family of the Leguminosae, especially the genus Glycine, very especially the species max (soybean), alfalfa, pea, lucerne, beans or peanut and many others; and the family of the Cruciferae (Brassicacae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

“Precise” with respect to the introduction of a donor DNA molecule in target region means that the sequence of the donor DNA molecule is introduced into the target region without any InDels, duplications or other mutations as compared to the unaltered DNA sequence of the target region that are not comprised in the donor DNA molecule sequence.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refer to a DNA sequence which when ligated to a nucleotide sequence of interest is capable of controlling the transcription of the nucleotide sequence of interest into RNA. Such promoters can for example be found in the following public databases http://www.grassius.org/grasspromdb.html, http://mendel.cs.rhul.ac.uk/mendel.php?topic=plantprom, http://ppdb.gene.nagoyau.ac.jp/cgi-bin/index.cgi. Promoters listed there may be addressed with the methods of the invention and are herewith included by reference. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective plant. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells (e.g., plants or plant pathogens like plant viruses). A plant specific promoter is a promoter suitable for regulating expression in a plant. It may be derived from a plant but also from plant pathogens or it might be a synthetic promoter designed by man. If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in the majority of plant tissues and cells throughout substantially the entire lifespan of a plant or part of a plant. Typically, constitutive promoters are capable of directing expression of a transgene in substantially any cell and any tissue.

Promoter specificity: The term “specificity” when referring to a promoter means the pattern of expression conferred by the respective promoter. The specificity describes the tissues and/or developmental status of a plant or part thereof, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought, wounding or infection.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In an especially preferred embodiment, the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the latter being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

“Target region” as used herein means the region close to, for example 10 bases, 20 bases, 30 bases, 40 bases, 50 bases, 60 bases, 70 bases, 80 bases, 90 bases, 100 bases, 125 bases, 150 bases, 200 bases or 500 bases or more away from the target site, or including the target site in which the sequence of the donor DNA molecule is introduced into the genome of a cell.

“Target site” as used herein means the position in the genome at which a double strand break or one or a pair of single strand breaks (nicks) are induced using recombinant technologies such as Zn-finger, TALEN, restriction enzymes, homing endonucleases, RNA-guided nucleases, RNA-guided nickases such as CRISPR/Cas nucleases or nickases and the like.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used inter-changeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention. A plant transformation vector is to be understood as a vector suitable in the process of plant transformation.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

### FIGURES

FIG. 1: Frequency of rice mono-allelic TIPS edited events with and without an InDel allele: paired Cas9 nickases vs Cas9 nuclease

### EXAMPLES

Chemicals and Common Methods

Unless indicated otherwise, cloning procedures carried out for the purposes of the present invention including restriction digest, agarose gel electrophoresis, purification of nucleic acids, Ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, Calif., USA) using the Sanger technology (Sanger et al., 1977). Unless described otherwise, chemicals and reagents were obtained from Sigma Aldrich (Sigma Aldrich, St. Louis, USA), from Promega (Madison, Wis., USA), Duchefa (Haarlem, The Netherlands) or Invitrogen (Carlsbad, Calif., USA). Restriction endonucleases were from New England Biolabs (Ipswich, Mass., USA) or Roche Diagnostics GmbH (Penzberg, Germany). Oligonucleotides were synthesized by Eurofins Eurofins Genomics (Ebersberg, Germany) or Integrated DNA Technologies (Coralville, Iowa, USA).

### Example 1: Screening of the Best gRNA and Donor DNA Combination for HDR-mediated Precise Gene Editing in Allohexaploid Wheat

Our approach for precise gene editing in wheat was based on screening first a set of different gRNA/donor DNA combinations at the scutellar callus level to identify the preferred gRNA/donor DNA combination to be used for the generation of edited plantlets.

In this example we describe that for the introduction of a specific single amino acid substitution (I1781L) into the coding sequence of the ACCase gene, we pre-screened 5 different gRNA/donor DNA combinations. Five different gRNAs were designed that guides the Cas9 to 5 different target sites near the target codon for the I1781L substitution. The sgRNA vectors pBAY02528 (SEQ ID NO: 5), pBAY02529 (SEQ ID NO: 6), pBAY02530 (SEQ ID NO: 7), pBAY02531 (SEQ ID NO: 8) and pBAY02532 ((SEQ ID NO: 9) each comprise a cassette for expression of the gRNA that can guide the Cas9 for the creation of a DSB at the target site TS1 sequence CTAGGTGTGGAGAACATACA-TGG (SEQ ID NO: 50), TS2 sequence GAAGGAGGATGGGCTAGGTG-TGG (SEQ ID NO: 51), TS3 sequence ATAGGCCCTAGAATAGGCAC-TGG (SEQ ID NO: 52), TS4 sequence CTCCTCATAGGCCCTAGAAT-AGG (SEQ ID NO: 53), TS5 CTATTGCCAGTGCCTATTCTAGG (SEQ ID NO: 54), respectively. Three donor DNA vectors were developed, pBAY02539 (SEQ ID NO: 13), pBAY02540 (SEQ ID NO: 14) and pBAY02541 (SEQ ID NO: 15) each including an 803 bp DNA fragment of Triticum aestivum, cv. Fielder subgenome B, ACCase gene containing the desired mutation (I1781L substitution). The 3 donor DNAs differ only in a few silent mutations to prevent cleavage of the donor DNA and the edited allele with the desired mutation (I1781L). The 3-bp (CTC) core sequence in each of the donor DNAs was flanked with an ˜400-bp left and right homologous arm, which are identical to the WT ACCase sequences of the subgenome B. The Cas9 expression pBAY02430 (SEQ ID NO: 1) comprises a Cas9 nuclease codon optimized for wheat and was under the control of the pUbiZm promoter and the 3′35S terminator. Plasmid DNA of a vector with the Cas9 nuclease, a gRNA, a donor DNA were mixed with the plasmid pIB26 (SEQ ID NO: 18) containing an egfp-bar fusion gene to allow selection on phosphinotricin (PPT) and screening for GFP fluorescence.

Immature embryos, 2-3 mm size, were isolated from sterilized ears of wheat cv. Fielder and bombarded using the PDS-1000/He particle delivery system was as described by Sparks and Jones (Cereal Genomics: Methods in Molecular biology, vol. 1099, Chapter 17). Following DNA mixtures were used for bombardment:
1) pBAY02430 (Cas9), pBAY02539 (donor DNA-1), pBAY02528 (gRNA1), pIB26
2) pBAY02430 (Cas9), pBAY02539 (donor DNA-1), pBAY02529 (gRNA2), pIB26
3) pBAY02430 (Cas9), pBAY02540 (donor DNA-2), pBAY02530 (gRNA3), pIB26
4) pBAY02430 (Cas9), pBAY02540 (donor DNA-2), pBAY02531 (gRNA4), pIB26
5) pBAY02430 (Cas9), pBAY02540 (donor DNA-2), pBAY02532 (gRNA5), pIB26
6) pBAY02430 (Cas9), pBAY02541 (donor DNA-3), pBAY02530 (gRNA3), pIB26
7) pBAY02430 (Cas9), pBAY02541 (donor DNA-3), pBAY02531 (gRNA4), pIB26
8) pBAY02430 (Cas9), pBAY02541 (donor DNA-3), pBAY02532 (gRNA5), pIB26

Bombarded immature embryos were transferred to non-selective callus induction medium for a few days, then moved to PPT containing selection media as described by Ishida et al. (Agrobacterium Protocols: Volume 1, Methods in Moleclar Biology, vol. 1223, Chapter 15). After 3 to 4 weeks, genomic DNA was extracted from scutellar calli from individual immature embryos for PCR analysis. Following primer pairs were designed for specific amplification of the edited ACCase gene: primer pair HT-18-111 Forward/HT-18-112 Reverse for donor DNA pBAY02539 (SEQ ID NO: 13), primer pair HT-18-113 Forward/HT-18-112 Reverse for donor DNA pBAY02540 (SEQ ID NO: 14) and donor DNA pBAY02541 (SEQ ID NO: 15) (Table 1). The efficiency of precise gene editing was highest when donor DNA-1 (pBAY02539) (SEQ ID NO: 13) was used in combination with gRNA1 pBAY02528 (SEQ ID NO: 5), With this gRNA/donor DNA combination 13% of the scutellar calli derived from individual immature embryos gave in the edit specific PCR, an amplification product of the expected size (Table 2).

For the generation of wheat plants with the ACCase (I1781L) mutation, we did a cobombardment of immature wheat embryos with DNA mixture 1) pBAY02430 (Cas9) (SEQ ID NO: 1) pBAY02539 (donor DNA-1) (SEQ ID NO: 13), pBAY02528 (gRNA1) (SEQ ID NO: 5), pIB26 (SEQ ID NO: 18) and we showed that wheat plants having the targeted AA susbsitution (I1781L) in one or more homeoalleles via indirect selection on PPT could be obtained with relatively high rates of success (see example 2). This demonstrates that a pre-screening of different gRNA/donor DNA combinations for precise HR-mediated gene editing in scutellar tissue from bombarded immature embryos as described in this example, allows a good prediction on the feasibility of generating wheat plants having the desired AA modification in one or more of the homeoalleles in allohexaploid wheat.

TABLE 1
Primers for edit-specific PCR (ACCasel1781L)

| | forward primer | | | reverse primer | | |
|---|---|---|---|---|---|---|
| | | | SEQ | | | SEQ |
| | | | ID | | | ID |
| donor DNA | name | sequence | NO | name | sequence | NO |
| pBAY02540 | HT-18- | GCTAGGTGTGGAGAACCTC | 30 | HT-18- | ACTTGCCCAGCACGAGGAAC | 29 |
| | 113 | | | 112 | | |
| PBAY02541 | HT-18- | GCTAGGTGTGGAGAACCTC | 30 | HT-18- | ACTTGCCCAGCACGAGGAAC | 29 |
| | 113 | | | 112 | | |
| pBAY02539 | HT-18- | GTTGGGCGTCGAGAACCTC | 28 | HT-18- | ACTTGCCCAGCACGAGGAAC | 29 |
| | 111 | | | 112 | | |

TABLE 2
Screening different gRNA/ donor DNA combinations for editing ACCasel1781L: No
of scutellar tissue samples positive in the edit PCR (ACCasel1781L)

| | | Samples | |
|---|---|---|---|
| | | with expected PCR fragment | |
| DNA delivery | # Samples analyzed | # Samples* | % |
| pBAY02430 (Cas9) + | 265 | 35 | 13.2 |
| PBAY02539 (donor DNA-1) + | | | |
| pBay02528 (gRNA1) + PIB26 | | | |
| PBAY02430 (Cas9) + | 275 | 5 | 1.8 |
| PBAY02539 (donor DNA-1) + | | | |
| pBay02529 (gRNA2) + PIB26 | | | |
| pBAY02430 (Cas9) + pBAY02540 | 137 | 1 | 0.7 |
| (donor DNA-2) + pBay02530 | | | |
| (gRNA3) + PIB26 | | | |
| pBAY02430 (Cas9) + pBAY02540 | 109 | 4 | 3.6 |
| (donor DNA-2) + pBay02531 | | | |
| (gRNA4) + PIB26 | | | |
| pBAY02430 (Cas9) + pBAY02540 | 122 | 0 | 0 |
| (donor DNA-2) + pBAY02532 | | | |
| (qRNA5) + PIB26 | | | |
| PBAY02430 (Cas9) + pBAY02541 | 103 | 0 | 0 |
| (donor DNA-3) + pBay02530 | | | |
| (gRNA3) + PIB26 | | | |
| PBAY02430 (Cas9) + pBAY02541 | 182 | 3 | 1.6 |
| (donor DNA-3) + pBay02531 | | | |
| (gRNA4) + PIB26 | | | |
| PBAY02430 (Cas9) + pBAY02541 | 112 | 0 | 0 |
| (donor DNA-3) + pBay02532 | | | |
| (gRNA5) + PIB26 | | | |

*only samples with the amplified edit specific PCR fragment with a concentration >2 ng/μL, have been considered as positive

### Example 2: Homology-Dependent Precise Gene Editing for the Introduction of the I1781L Mutation in the ACCase (Acetyl-CoA Carboxylase) Gene of Allohexaploid Wheat by a Cas9 Nuclease

We demonstrated that by using a Cas9 nuclease and a pre-screened gRNA/donor DNA combination for its capability of potential HR-mediated precise gene editing in allohexaploid wheat as described in example 1, the desired mutation can be introduced in the target codon in one or more homeoalleles. The sgRNA vector pBAY02528 (SEQ ID NO: 5) comprises a cassette for expression of the gRNA1 that guides the Cas9 nuclease for the creation of a DSB at the target site TS1 sequence CTAGGTGTGGAGAACATACA-TGG (SEQ ID NO: 50) which is positioned over the target codon. The donor DNA pBAY2539 was designed for the introduction of 2 base substitutions at the target codon (ATA to CTC) leading to the I1781L change at the protein level. The donor DNA includes an 803 bp DNA fragment of Triticum aestivum, cv. Fielder subgenome B, ACCase gene containing the desired mutation (I1781L substitution). The donor DNA contains also some other silent mutations to prevent cleavage of the donor DNA and the edited allele with the desired mutation (I1781L). The 3-bp (CTC) core sequence in the donor DNA was flanked with an ˜400-bp left and right homologous arm, which are identical to the WT ACCase sequences of the subgenome B.

Immature embryos, 2-3 mm size, were isolated from sterilized ears of wheat cv. Fielder and bombarded using the PDS-1000/He particle delivery system as described by Sparks and Jones (Cereal Genomics: Methods in Molecular biology, vol. 1099, Chapter 17). Plasmid DNA of vectors pBAY02430 (Cas9 nuclease) (SEQ ID NO: 1), pBAY02528 (gRNA) (SEQ ID NO: 5), pBAY02539 (donor DNA) (SEQ ID NO: 13) were mixed with the plasmid pIB26 (SEQ ID NO: 18). The vector pIB26 (SEQ ID NO: 18) contains an egfp-bar fusion gene under control of the 35S promoter. Bombarded immature embryos were transferred to non-selective callus induction medium for 1-2 weeks, then moved to PPT containing selection media and PPT resistant calli were selected and transferred to regeneration media for shoot formation as described by Ishida et al. (Agrobacterium Protocols: Volume 1, Methods in Molecular Biology, vol. 1223, Chapter 15).

All plants developed from one immature embryo were treated as a pool. Genomic DNA was extracted from pooled leaf samples and a primer set (HT-18-111 Forward (SEQ ID NO: 28)/HT-18-112 Reverse (SEQ ID NO: 29)) was designed for specific amplification of the edited ACCase gene. The plantlets in a pool that gave the expected PCR fragment in this 1^{st} edit specific PCR, were then transferred to individual tubes and further analyzed by PCR using primer set HT-18-111 (SEQ ID NO: 28)/HT-18-112 (SEQ ID NO: 29) and by deep sequencing. For 9 experiments a total of 337, 326, 415, 322, 350, 329, 261, 361 and 362 embryos were bombarded with a mixture of plasmid DNA of pBAY02430 (Cas9 nuclease) (SEQ ID NO: 1), pBAY02528 (gRNA) (SEQ ID NO: 5), pBAY02539 (donor DNA) (SEQ ID NO: 13) and pIB26 (SEQ ID NO: 18). In these 9 experiments, phosphinotricin (PPT) tolerant shoot regenerating calli were obtained from in total 132, 172, 111, 177, 107, 166, 122, 244 and 279 immature embryos. Specific amplification of the edited ACCase gene was observed in 8, 17, 15, 9, 16, 7, 6, 9 and 8 pooled leaf samples. A 2^{nd} edit specific PCR was performed on in total 51, 62, 66, 33, 49, 25, 35, 42 and 31 individual plants derived from 8, 15, 15, 8, 16, 7, 6, 9 and 8 plantlet pools scored as positive in the 1^{st} edit PCR and specific amplification of the edited ACCase gene was observed in 16, 28, 12, 25, 19, 19, 13, 21 and 12 individual plantlets derived from 6, 11, 8, 7, 10, 7, 4, 8 and 8 plantlet pools, respectively (Table 3). As each plantlet pool is derived from a single immature embryo, all plantlets derived from a single immature embryo (plantlet pool) are considered as an independent edited event, although we can't exclude that there might be multiple independent edited events between individual shoots derived from a single immature embryo scored as positive in the 2^{nd} edit PCR. On one plant from each event scored as positive in the 2^{nd} edit PCR, deep sequencing was performed. The region surrounding the intended target site was PCR amplified with Q5 High-Fidelity polymerase (M0492L) by means of nested PCR. For the 1^{st} PCR primer pair HT-18-162 (SEQ ID NO: 34)/HT-18-112 (SEQ ID NO: 29) was used; these primers were positioned outside the homology arms of the donor DNA for the amplification of a 1736 bp fragment. For the nested PCR to amplify a region of a 386 bp for NGS, primer pair HT-18-048 (SEQ ID NO: 19)/HT-18-053 (SEQ ID NO: 21) was used.

We assessed editing frequency by calculating the percentage of sequence reads showing evidence for presence of the desired mutations (AA substitution) at the target codon as directed by the donor DNA, as a proportion of the total number of reads. These data are summarized in Table 4 showing the % of precisely edited reads with the desired mutation (the I1781L substitution) and the % of WT reads based on the total number of reads for 64 plantlets from 59 independent events. The control sample from plantlet TMTA0136-Ctr10001-01$002 derived from a non-bombarded immature embryo showed ˜100% WT reads and no precisely edited reads, as expected.

These deep sequencing analysis data showed precise gene editing by homologous recombination (HR) of one up to 4 alleles of the native ACCase gene in allohexaploid wheat. HR-mediated precise donor resulting in the desired AA substitution and the introduction of additional silent mutations as directed by the donor DNA, was further confirmed by Sanger sequencing of cloned PCR fragments. On 11 of these events analyzed by deep sequencing, PCR amplification over the target region with primer pair HT-18-162 Forward (SEQ ID NO: 34)/HT-18-112 (SEQ ID NO: 29) Reverse, cloning and Sanger sequencing was performed for subgenomic characterization. Between 52 to 96 clones were sequenced per event. These data are summarized in Table 5 and show that plants with precisely edited allele(s) contain most often also allele(s) with NHEJ-derived InDels and sometimes also WT allele(s). These TO plants have been transferred to the greenhouse for seed production. Plants from independent events with the precise edited allele on different subgenomes can be crossed to create plants with the desired AA modification in e.g. all 3 homeologous copies of the ACCase gene, and the undesired alleles with NHEJ-derived Indels being removed by progeny segregation.

TABLE 3
Number of ACCase I1781L edited plantlets
based on edit PCR analysis

| | | | | # | # individual |
|---|---|---|---|---|---|
| | | | | plantlets | plantlets |
| | | | | tested | positive in |
| | | | | in 2nd | 2nd edit |
| | | | # positive | edit PCR, | PCR, |
| | | | leaf | (derived | (derived |
| | # bom- | PPT^{R} shoot | pools in | from # | from # |
| Exp | barded | regenerating | 1st edit | leaf | of leaf |
| n^{o} | embryos | calli | PCR | pools*) | pools*) |
| 1 | 337 | 132 | 8 | 51 (8) | 16 (6) |
| 2 | 326 | 172 | 17 | 62 (15) | 28 (11) |
| 3 | 415 | 111 | 15 | 66 (15) | 12 (8) |
| 4 | 322 | 177 | 9 | 33 (8) | 25 (7) |
| 5 | 350 | 107 | 16 | 49 (16) | 19 (10) |
| 6 | 329 | 166 | 7 | 25 (7) | 19 (7) |
| 7 | 261 | 122 | 6 | 35 (6) | 13 (4) |
| 8 | 361 | 244 | 9 | 42 (9) | 21 (8) |
| 9 | 362 | 279 | 8 | 31 (8) | 12 (8) |

*each leaf pool is derived from one immature embryo

TABLE 4
Percent (%) precisely edited reads at the Acetyl-
CoA carboxylase target locus (ACCase I1781L)
in individual plantlets from independent events
scored as positive in the 2nd edit PCR

| | NGS on individual | | | |
|---|---|---|---|---|
| | shoots from independant | | | |
| | events, positive in | | | |
| | the 2nd edit PCR | | | |
| | Target | % edit | % WT | Sanger se- |
| Event name | reads | reads | reads | quencing |
| TMTA0136-Ctrl0001-01$002 | 40709 | 0 | 99.78 | |
| TMTA0131-0003-B01-04$001 | 41239 | 27.75 | 0.05 | X |
| TMTA0131-0030-B01-02$001 | 42137 | 20.53 | 0.07 | |
| TMTA0131-0089-B01-01$001 | 40069 | 16.78 | 53.99 | X |
| TMTA0131-0091-B01-01$001 | 36830 | 23.25 | 17.63 | X |
| TMTA0132-0005-B01-02$001 | 40995 | 9.19 | 51.37 | |
| TMTA0132-0038-B01-01$001 | 42379 | 8 | 59.05 | |
| TMTA0132-0058-B01-02$001 | 43429 | 21.39 | 0.05 | |
| TMTA0132-0075-B01-03$001 | 50651 | 16.35 | 0.04 | |
| TMTA0132-0079-B01-01$001 | 40691 | 19.22 | 32.75 | X |
| TMTA0132-0082-B01-01$001 | 102234 | 21.17 | 0.01 | |
| TMTA0132-0083-B01-01$001 | 44100 | 20.42 | 0 | |
| TMTA0132-0084-B01-01$001 | 34262 | 19.75 | 17.78 | |
| TMTA0132-0130-B01-02$001 | 28768 | 21.25 | 0.02 | |
| TMTA0132-0138-B01-02$001 | 34718 | 20.91 | 0 | |
| TMTA0136-0013-B01-01$001 | 42346 | 60.42 | 0 | X |
| TMTA0136-0039-B01-02$001 | 41189 | 20.05 | 78.93 | X |
| TMTA0136-0055-B01-03$001 | 33875 | 21.23 | 0.03 | |
| TMTA0136-0081-B01-01$001 | 49956 | 19.38 | 13.46 | |
| TMTA0136-0108-B01-01$001 | 51522 | 27.33 | 0.01 | X |
| TMTA0136-0110-B01-01$001 | 52048 | 16.69 | 0 | |
| TMTA0137-0016-B01-02$001 | 19342 | 17.06 | 14.67 | |
| TMTA0137-0016-B01-04$001 | 19125 | 16.88 | 14.27 | |
| TMTA0137-0017-B01-03$001 | 10598 | 17.42 | 14.87 | |
| TMTA0137-0018-B01-04$001 | 20526 | 16.23 | 15.17 | |
| TMTA0137-0105-B01-01$001 | 23270 | 4.62 | 72.13 | |
| TMTA0137-0107-B01-01$001 | 27218 | 18.93 | 21.18 | |
| TMTA0137-0155-B01-01$001 | 10940 | 25.43 | 0 | |
| TMTA0138-0025-B01-03$001 | 33577 | 19.53 | 16.75 | |
| TMTA0138-0028-B01-01$001 | 40346 | 16.09 | 0 | |
| TMTA0138-0034-B01-01$001 | 35875 | 30.22 | 0.07 | |
| TMTA0138-0035-B01-01$001 | 129047 | 31.98 | 0.01 | X |
| TMTA0138-0041-B01-01$001 | 44938 | 18.35 | 0.02 | |
| TMTA0138-0049-B01-01$001 | 45611 | 21.59 | 0.04 | |
| TMTA0138-0058-B01-03$001 | 43272 | 16.53 | 12.43 | |
| TMTA0138-0059-B01-02$001 | 39400 | 24.16 | 17.8 | |
| TMTA0138-0072-B01-04$001 | 34732 | 20.41 | 11.3 | |
| TMTA0138-0083-B01-01$001 | 31915 | 14.98 | 12.2 | |
| TMTA0140-0004-B01-04$001 | 40316 | 22.64 | 0.02 | |
| TMTA0140-0007-B01-01$001 | 33213 | 17.7 | 23.4 | |
| TMTA0140-0013-B01-03$001 | 45408 | 20.8 | 0 | |
| TMTA0140-0048-B01-01$001 | 36021 | 65.03 | 3.94 | X |
| TMTA0140-0050-B01-01$001 | 53818 | 32.57 | 0.04 | X |
| TMTA0143-0001-B01-01$001 | 35829 | 24.15 | 0.03 | |
| TMTA0143-0086-B01-01$001 | 107131 | 34.64 | 0.05 | x |
| TMTA0147-0001-B01-02$001 | 34822 | 11.36 | 18.7 | |
| TMTA0171-0047-B01-02$001 | 26724 | 11.18 | 31.67 | |
| TMTA0171-0053-B01-01$001 | 27004 | 12.49 | 23.24 | |
| TMTA0171-0053-B01-03$001 | 37877 | 11.17 | 26.94 | |
| TMTA0171-0080-B01-02$001 | 26062 | 7.11 | 45.67 | |
| TMTA0171-0086-B01-03$001 | 21361 | 15.46 | 0.01 | |
| TMTA0171-0086-B01-05$001 | 44053 | 16.87 | 20.33 | |
| TMTA0171-0134-B01-02$001 | 29626 | 9.21 | 0 | |
| TMTA0171-0220-B01-01$001 | 29826 | 27.56 | 16.94 | |
| TMTA0171-0220-B01-03$001 | 35492 | 29.21 | 16.84 | |
| TMTA0172-0001-B01-04$001 | 37739 | 12.56 | 15.61 | |
| TMTA0172-0180-B01-02$001 | 36540 | 26.34 | 16.21 | |
| TMTA0172-0180-B01-05$001 | 43100 | 25.22 | 14.44 | |
| TMTA0172-0183-B01-01$001 | 39955 | 11.93 | 0.01 | |

TABLE 5
The ACCase locus genotypes in 11 T0 plants from independent events by Sanger
sequencing of cloned PCR fragments. Precise edit refers to the presence of a precisely
edited ACCase allele with the desired AA substitution and the additional silent mutations as
directed by the donor DNA, InDel refers to the presence of a NHEJ mutation and WT refers
to the presence of a WT native ACCase sequence. The numbers before Precise Edit, WT,
InDei indicate the frequency at which the 3 different versions of the ACCase allele were
identified.

| | NCS | | Sanger sequencing | | |
|---|---|---|---|---|---|
| Event | edit % | WT % | A | B | D |
| TMTA0131-0003- | 27.75 | 0.05 | 45 indel | 14 precise | no reads |
| B01-04$001 | | | | edit; 25 indel | |
| TMTA0131-0089- | 16.78 | 53.99 | 28 WT | 7 WT; 16 | 11 precise edit; 11 |
| B01-01$001 | | | | indel | indel; 6WT |
| TMTA0131-0091- | 23.25 | 17.63 | 29 indel | 17 precise | 12 indel; 12WT |
| B01-01$001 | | | | edit; 12 indel | |
| TMTA0132-0079- | 19.22 | 32.75 | 12 precise | 9 indel; 10 | 18 indel; 12 indel |
| B01-01$001 | | | edit; 21 WT | WT | |
| TMTA0136-0039- | 20.05 | 78.93 | 34 WT | 11 precise | 1 precise edit; 30 WT |
| B01-02$001 | | | | edit; 24 WT | |
| TMTA0136-0108- | 27.33 | 0.01 | 18 precise | 13 indel | 18 indel |
| B01-01$001 | | | edit; 17 indel | | |
| TMTA0138-0035- | 31.98 | 0.01 | 21 precise | 12 indel; 20 | 14 precise edit |
| B01-01$001 | | | edit; 17 indel | indel | |
| TMTA0140-0048- | 65.03 | 3.94 | 28 indel | 33 precise | 15 precise edit |
| B01-01$001 | | | | edit; 6 indel | |
| TMTA0140-0050- | 32.57 | 0.04 | 10 precise | 7 precise edit; | 14 precise edit; 11 |
| B01-01$001 | | | edit; 13 indel | 22 indel | indel |
| TMTA0143-0086- | 34.64 | 0.05 | 14 precise | 19 precise | 23 indel |
| B01-01$001 | | | edit; 9 indel | edit; 15 indel | |
| TMTA0136-0013- | 59 | 6.79 | 8 precise edit | 31 precise edit | 13 indel |
| B01-01$001 | | | | | |

### Example 3: Homology-Dependent Precise Gene Editing for the Introduction of the I1781L Mutation in the ACCase (Acetyl-CoA Carboxylase) Gene of Allohexaploid Wheat by a Paired Cas9 Nickase

The following example describes homology-dependent precise gene editing for the introduction of the I1781L mutation in the ACCase (Acetyl-CoA carboxylase) gene of allohexaploid wheat by a paired Cas9 nickase. By using a Cas9 nickase and 2 sgRNAs leading the SpCas9 nickase to 2 target sites (TS1, T2) within proximity of each other on opposite strands and in close proximity of the target codon ACCase 11781, and a donor DNA, the desired mutation can be efficiently introduced in the target codon. A Cas9 nickase expression vector pBay02734 (SEQ ID NO: 3) was constructed. The Cas9 nickase by mutation of Aspartic acid to Alanine at position 10 within the RuvC domain (the D10A mutation), was codon optimized for wheat and was under the control the pUbiZm promoter and the 3′35S terminator. Two sgRNAs were designed for targeting all gene copies on the 3 wheat subgenomes A, B and D and for the generation of 32 bp 3′ overhangs spanning the target codon. The sgRNA vector pBAY02528 (SEQ ID NO: 5) comprises a cassette for expression of the gRNA1 that can guide the Cas9 nickase for the creation of a nick at the target site TS1 sequence CTAGGTGTGGAGAACATACA-TGG (SEQ ID NO:50). The sgRNA vector pBAY02531 comprises a cassette for expression of the gRNA2 targeting target site TS2 sequence CTCCTCATAGGCCCTAGAAT-AGG (SEQ ID NO:53). A donor DNA pBAY02540 (SEQ ID NO: 14) was designed for the introduction of 2 base substitutions at the target codon (ATA to CTC) leading to the I1781L change at the protein level. The donor DNA includes an 803 bp DNA fragment of Triticum aestivum, cv. Fielder subgenome B, ACCase gene containing the desired mutation (I1781L substitution). The donor DNA contains also some other silent mutations to prevent cleavage of the donor DNA and the edited allele with the desired mutation (I1781L). The 3-bp (CTC) core sequence in the donor DNA was flanked with an ˜400-bp left and right homologous arm, which are identical to the WT ACCase sequences of the subgenome B.

Immature embryos, 2-3 mm size, were isolated from sterilized ears of wheat cv. Fielder and bombarded using the PDS-1000/He particle delivery system as described by Sparks and Jones (Cereal Genomics: Methods in Molecular biology, vol. 1099, Chapter 17). Plasmid DNA of vectors pBAY02734 (Cas9 nickase) (SEQ ID NO: 3), pBAY02528 (gRNA1) (SEQ ID NO: 5), pBAY02531 (gRNA2) (SEQ ID NO:8), pBAY02540 (donor DNA) (SEQ ID NO: 14) were mixed with the plasmid pIB26 (SEQ ID NO: 18). The vector pIB26 (SEQ ID NO: 18) contains an egfp-bar fusion gene under control of the 35S promoter. Bombarded immature embryos were transferred to non-selective callus induction medium for 1-2 weeks, then moved to PPT containing selection media and PPT resistant calli were selected and transferred to regeneration media for shoot formation as described by Ishida et al. (Agrobacterium Protocols: Volume 1, Methods in Molecular Biology, vol. 1223, Chapter 15). All plants developed from one immature embryo were treated as a pool. Genomic DNA was extracted from pooled leaf samples and a primer set (HT-18-113 Forward/HT-18-112 Reverse (SEQ ID NOs: 30; 29)) was designed for specific amplification of the edited ACCase gene. The plantlets in a pool that gave the expected PCR fragment in this 1^{st} edit specific PCR, were then transferred to individual tubes and further analyzed by PCR using primer set HT-18-113/HT-18-112 (SEQ ID NOs: 30; 29) and by deep sequencing. For 6 experiments a total of 358, 423, 365, 355, 409, and 395 embryos were bombarded with a mixture of plasmid DNA of pBAY02734 (Cas9 nickase) (SEQ ID NO: 3), pBAY02528 (gRNA1) (SEQ ID NO: 5), pBAY02531 (gRNA2) (SEQ ID NO: 8), pBAY02540 (donor DNA) (SEQ ID NO: 14) and pIB26 (SEQ ID NO: 18). In these 6 experiments, phosphinotricin (PPT) tolerant shoot regenerating calli were obtained from in total 195, 163, 192, 181, 268 and 190 immature embryos. Specific amplification of the edited ACCase gene was observed in 13, 6, 44, 22, 21 and 22 pooled leaf samples. A 2^{nd} edit specific PCR was performed on in total 45, 20, 258, 64, 94, 93 individual plants derived from 11, 5, 39, 17, 16 and 20 plantlet pools scored as positive in the 1^{st} edit PCR. Specific amplification of the edited ACCase gene was observed in 22, 18, 93, 41, 18 and 35 individual shoots derived from 11, 5, 33, 14, 12 and 17 plantlet pools, respectively (Table 6). As each plantlet pool is derived from a single immature embryo, all plantlets derived from a single immature embryo (plantlet pool) are considered as an independent edited event, although we can't exclude that there might be multiple independent edited events between individual shoots derived from a single immature embryo scored as positive in the 2^{nd} edit PCR. On one plant from each event scored as positive in the 2^{nd} edit PCR, deep sequencing was performed. The region surrounding the intended target site was PCR amplified with Q5 High-Fidelity polymerase (M0492L) by means of nested PCR. For the 1^{st} PCR primer pair HT-18-162/HT18-112 (SEQ ID NO 34; 29) was used; these primers were positioned outside the homology arms of the donor DNA for the amplification of a 1736 bp fragment. For the nested PCR to amplify a region of a 386 bp for NGS, primer pair HT-18-048/HT-18-053 (SEQ ID NOs: 19, 21) was used.

We assessed editing frequency by calculating the percentage of sequence reads showing evidence for presence of the desired I1781L mutation at the target codon, as a proportion of the total number of reads. These data are summarized in Table 7 showing for 57 plantlets, all derived from independent events, the total number of reads, the % of reads with the desired mutation (the I1781L substitution), the % of reads with the desired mutation and all silent mutations as present in the donor DNA, and the % of WT reads. These deep sequencing analysis data showed that one up to 4 alleles of the native ACCase gene in allohexaploid wheat contain the desired I1781L substitution. These data further show that in plants with the desired AA substitution not all silent mutations from the repair DNA have been always introduced. The silent mutations were positioned around target site TS2 (gRNA2). These data further show that ˜50% (28/57) of the plants with allele(s) with the desired edit (I1781L) don't contain reads with NHEJ-derived InDels. In the other 50% the number of reads with NHEJ-derived InDels was sometimes very low. In contrast by using a CRISPR/Cas9 nuclease instead of a CRISPR/Cas nickase, 98-100% of the events with one or more precisely edited alleles also contain allele(s) with NHEJ-derived InDels (Table 4). The absence of alleles with Indels in events with precisely edited alleles by making use of a nickase will make it easier to study the dosage effects of the performance impact of the precisely edited allele(s) as for one or more of the wheat subgenomes (A,B,D) plants homozygous (HH), hemizygous (Hh) and WT (hh) for the precise edit will become available already in the T1 generation for further performance evaluation. Plants from independent events with the precise edited allele on different subgenomes can be crossed to create plants with the desired AA modification in e.g. all 3 homeologous copies of the target gene.

TABLE 6
Number of ACCase I1781L edited plantlets by the use of
a Cas9 paired nickase based on edit PCR analysis

| | | | | # plant- | |
|---|---|---|---|---|---|
| | | | | lets tested | # individual |
| | | | | in 2nd | plantlets |
| | | | # positive | edit PCR, | positive |
| | | | leaf | (derived | in 2nd |
| | # bom- | PPT^{R} shoot | pools in | from # | edit PCR, (de- |
| Exp | barded | regenerating | 1st edit | leaf | rived from # |
| n^{o} | embryos | calli | PCR | pools*) | of leaf pools*) |
| 1 | 358 | 195 | 13 | 45 (11) | 22 (11) |
| 2 | 423 | 163 | 6 | 20 (5) | 18 (5) |
| 3 | 365 | 192 | 44 | 258 (39) | 93 (33) |
| 4 | 355 | 181 | 22 | 64 (17) | 41 (14) |
| 5 | 409 | 268 | 21 | 125 (19) | 18 (12) |
| 6 | 395\| | 190 | 22 | 118 (22) | 35 (17) |

TABLE 7
Percent (%) precisely edited reads at the Acetyl-CoA carboxylase target locus
(ACCase I1781L) in individual plantlets from independent events scored as positive in the
2^{nd} edit PCR

| | NGS on individual shoots from inde- | | | | |
|---|---|---|---|---|---|
| | pendent events, positive in the 2nd | | | | |
| | edit PCR | | | | |
| | | | % edit | | |
| | | | I > L + all | | |
| | | % edit | silent | | no |
| | Target | I > L | mutations | | InDel |
| Event name | reads | reads | reads | % WT | reads |
| TMTA0252-0018-B01-01$001 | 22708 | 31.72 | 0 | 29.17 | |
| TMTA0252-0020-B01-03$001 | 58416 | 14.89 | 14.29 | 40.9 | |
| TMTA0252-0022-B01-04$001 | 52965 | 23.84 | 0 | 71.26 | X |
| TMTA0252-0038-B01-01$001 | 54433 | 21.98 | 21.04 | 56.1 | |
| TMTA0252-0072-B01-03$001 | 53496 | 18.55 | 0 | 76.46 | X |
| TMTA0253-0060-B01-01$001 | 37901 | 17.46 | 16.66 | 73.37 | |
| TMTA0254-0001-B01-03$001 | 53446 | 29.83 | 27.68 | 65.81 | X |
| TMTA0254-0002-B01-01$001 | 51254 | 18.46 | 0 | 76.5 | X |
| TMTA0254-0009-B01-02$001 | 56029 | 41.18 | 21.06 | 53.65 | X |
| TMTA0254-0010-B01-03$001 | 51141 | 41.01 | 20.72 | 53.37 | X |
| TMTA0254-0045-B01-01$001 | 39511 | 21.12 | 19.94 | 73.14 | X |
| TMTA0254-0054-B02-01$001 | 41727 | 20.19 | 0 | 72.78 | X |
| TMTA0254-0068-B01-01$001 | 43282 | 15.66 | 0 | 56.99 | |
| TMTA0254-0070-B01-01$001 | 17115 | 24.29 | 23.32 | 69.83 | X |
| TMTA0254-0071-B01-05$001 | 41360 | 17.06 | 16.02 | 76.96 | X |
| TMTA0254-0080-B02-03$001 | 29495 | 12.81 | 0 | 47.2 | |
| TMTA0254-0082-B01-01$001 | 40045 | 15.96 | 0 | 51.4 | |
| TMTA0254-0087-B01-01$001 | 40672 | 18.24 | 0 | 76.34 | X |
| TMTA0254-0105-B01-02$001 | 42879 | 22.12 | 21.27 | 47.7 | |
| TMTA0254-0110-B01-01$001 | 42238 | 20.13 | 0 | 75.73 | X |
| TMTA0254-0111-B01-01$001 | 42935 | 45.59 | 24.6 | 51.26 | X |
| TMTA0254-0120-B01-01$001 | 36683 | 18.94 | 18.13 | 51.47 | |
| TMTA0254-0120-B01-07$001 | 39382 | 17.6 | 16.9 | 51.12 | |
| TMTA0254-0132-B01-03$001 | 39999 | 38.98 | 37.3 | 54.96 | X |
| TMTA0254-0139-B01-03$001 | 43059 | 41.63 | 31.03 | 35.57 | |
| TMTA0255-0073-B01-01$001 | 42027 | 13.74 | 0 | 81.48 | X |
| TMTA0255-0080-B01-01$001 | 43476 | 63.73 | 36.67 | 26.9 | X |
| TMTA0255-0098-B01-01$001 | 48254 | 18.38 | 0 | 52.77 | |
| TMTA0255-0110-B01-01$001 | 38849 | 30.94 | 0.17 | 64.5 | |
| TMTA0255-0112-B01-03$001 | 48472 | 26.23 | 25.19 | 51.21 | |
| TMTA0255-0133-B01-01$001 | 1890532 | 23.83 | 23.2 | 24.45 | |
| TMTA0257-0104-B01-02$001 | 640098 | 13.8 | 0 | 62.47 | |
| TMTA0252-0078-B02-01$001 | 76441 | 14.87 | 14.17 | 36.79 | |
| TMTA0252-0109-B01-01$001 | 69453 | 21.27 | 20.2 | 72.85 | |
| TMTA0252-0142-B01-01$001 | 71863 | 20.43 | 19.62 | 47.18 | |
| TMTA0252-0156-B01-02$001 | 65565 | 15.87 | 0 | 78.3 | X |
| TMTA0254-0177-B01-01$001 | 67618 | 15.35 | 14.35 | 60.98 | |
| TMTA0254-0186-B01-07$001 | 67449 | 28.66 | 28.11 | 14.79 | |
| TMTA0254-0187-B01-04$001 | 70634 | 21.63 | 20.46 | 71.54 | X |
| TMTA0255-0012-B02-03$001 | 74277 | 19.47 | 18.54 | 52.18 | |
| TMTA0255-0040-B01-01$001 | 64076 | 21.02 | 0 | 74.27 | X |
| TMTA0255-0061-B01-01$001 | 69062 | 21.75 | 20.54 | 72.68 | X |
| TMTA0257-0040-B01-08$001 | 69229 | 13.99 | 13.37 | 58.78 | |
| TMTA0257-0074-B02-01$001 | 72358 | 11.77 | 11.07 | 70.52 | |
| TMTA0257-0133-B01-06$001 | 71008 | 13.93 | 13.35 | 57.74 | |
| TMTA0257-0169-B01-02$001 | 73796 | 4.42 | 4.2 | 90.43 | X |
| TMTA0257-0208-B01-02$001 | 65922 | 20.94 | 19.58 | 75.39 | X |
| TMTA0258-0019-B01-02$001 | 67969 | 13.19 | 0 | 38.41 | |
| TMTA0258-0044-B01-05$001 | 66375 | 21.75 | 21.26 | 32.46 | |
| TMTA0258-0051-B02-02$001 | 66099 | 13.93 | 13.21 | 80.61 | X |
| TMTA0258-0079-B01-01$001 | 68208 | 15.94 | 0 | 56.84 | |
| TMTA0258-0084-B01-04$001 | 32557 | 21.81 | 20.68 | 70.33 | X |
| TMTA0258-0105-B01-01$001 | 70097 | 18.99 | 18.09 | 73.83 | X |
| TMTA0258-0111-B02-03$001 | 66455 | 29.7 | 28.29 | 65.05 | X |
| TMTA0258-0161-B01-01$001 | 69256 | 22.16 | 20.87 | 71 | X |
| TMTA0258-0166-B01-07$001 | 69820 | 21.65 | 20.31 | 72.56 | X |
| TMTA0258-0170-B02-05$001 | 74311 | 13.72 | 0 | 69.3 | |

### Example 4: Homology-Dependent Precise Gene Editing for the Introduction of the A2004V Mutation in the ACCase (Acetyl-CoA Carboxylase) Gene of Allohexaploid Wheat by a Cas9 Nuclease

By using a Cas9 nuclease and a pre-screened gRNA/donor DNA combination for its capability of potential HR-mediated precise gene editing capability in allohexaploid wheat as described in example 1, we recovered edited wheat plants having the desired amino acid substitution A2004V in one or more alleles of the ACCase gene by HR-mediated donor of a targeted DSB and via indirect selection for resistance to PPT. The sgRNA vector pBAY02524 (SEQ ID NO: 10) comprises a cassette for expression of the gRNA that guides the Cas9 nuclease for the creation of a DSB at the target site TS sequence TTCCTCGTGCTGGGCAAGTC-TGG (SEQ ID NO: 55) which is positioned close upstream of the target GCT codon. The donor DNA pBAY02536 (SEQ ID NO: 16) was designed for the introduction of 2 base substitutions at the target codon (GCT to GTC) leading to the A2004 change at the protein level. The donor DNA includes an 787 bp DNA fragment of Triticum aestivum, cv. Fielder subgenome B, ACCase gene containing the desired mutation (A2004V substitution). The donor DNA contains also some other silent mutations to prevent cleavage of the donor DNA and the edited allele with the desired mutation (A2004V). The 3-bp (GTC) core sequence in the donor DNA was flanked with an ˜390-bp left and right homologous arm, which are identical to the WT ACCase sequences of the subgenome B. Immature embryos, 2-3 mm size, were isolated from sterilized ears of wheat cv. Fielder and bombarded using the PDS-1000/He particle delivery system as described by Sparks and Jones (Cereal Genomics: Methods in Molecular biology, vol. 1099, Chapter 17). Plasmid DNA of vectors pBAY02430 (Cas9 nuclease) (SEQ ID NO: 1), pBAY02524 (gRNA) (SEQ ID NO: 10), pBAY02536 (donor DNA) (SEQ ID NO: 16) were mixed with the plasmid pIB26 (SEQ ID NO: 18). The vector pIB26 (SEQ ID NO: 18) contains an egfp-bar fusion gene under control of the 35S promoter. Bombarded immature embryos were transferred to non-selective callus induction medium for 1-2 weeks, then moved to PPT containing selection media and PPT resistant calli were selected and transferred to regeneration media for shoot formation as described by Ishida et al. (Agrobacterium Protocols: Volume 1, Methods in Molecular Biology, vol. 1223, Chapter 15).

All plants developed from one immature embryo were treated as a pool. Genomic DNA was extracted from pooled leaf samples and a primer pair (HT-18-101 Forward (SEQ ID NO: 25)/HT-18-102 Reverse (SEQ ID NO: 26)) was designed for specific amplification of the edited ACCase gene. The plantlets in a pool that gave the expected PCR fragment in this 1^{st} edit specific PCR, were then transferred to individual tubes and further analyzed by PCR using primer set HT-18-101 Forward (SEQ ID NO: 25)/HT-18-102 Reverse (SEQ ID NO: 26) and by deep sequencing. For 4 experiments a total of 382, 424, 401 and 375 embryos were bombarded with a mixture of plasmid DNA of pBAY02430 (Cas9 nuclease) (SEQ ID NO: 1), pBAY02524 (gRNA1) (SEQ ID NO: 10), pBAY02536 (donor DNA-1) (SEQ ID NO: 16) and pIB26 (SEQ ID NO: 18). In these 4 experiments, phosphinotricin (PPT) tolerant shoot regenerating calli were obtained from in total 107, 326, 341 and 300 immature embryos. Specific amplification of the edited ACCase gene was observed in 2, 28, 7 and 5 pooled leaf samples. A 2^{nd} edit specific PCR was performed on in total 14, 259, 29 and 40 individual plants derived from 2, 27, 6 and 5 plantlet pools scored as positive in the 1^{st} edit PCR and specific amplification of the edited ACCase gene was observed in 7, 58, 7 and 7 individual plantlets, derived from 2, 23, 3 and 6 plantlet pools, respectively (Table 8). As each plantlet pool is derived from a single immature embryo, all plantlets derived from a single immature embryo (plantlet pool) are considered as an independent edited event, although we can't exclude that there might be multiple independent edited events between individual shoots derived from a single immature embryo scored as positive in the 2^{nd} edit PCR. On plants from independent events scored as positive in the 2^{nd} edit PCR, deep sequencing was performed. For the 1^{st} PCR primer pair HT-18-101 (SEQ ID NO: 25)/HT-18-110 (SEQ ID NO: 27) was used; these primers were positioned outside the homology arms of the donor DNA for the amplification of a 1313 bp fragment. For the nested PCR to amplify a region of 348 bp for NGS, primer pair HT-18-051 (SEQ ID NO: 20)/HT-18-054 (SEQ ID NO: 22) was used. These data showed that we have recovered plants with one or two alleles precisely edited with the desired AA substitution A2004V (Table 9).

TABLE 8

| | | | | # plantlets | |
|---|---|---|---|---|---|
| | | | | tested in | # individual |
| | | | | 2nd edit | plantlets |
| | | | | PCR, | positive |
| | | | # positive | (derived | in 2nd edit |
| | # | PPT^{R} shoot | leaf pools | from # | PCR, (derived |
| Exp | bombarded | regenerating | in 1st edit | leaf | from # of leaf |
| n° | embryos | calli | PCR | pools*) | pools*) |
| 1 | 382 | 107 | 2 | 14 (2) | 7 (2) |
| 2 | 424 | 326 | 28 | 259 (27) | 58 (23) |
| 3 | 401 | 341 | 7 | 29 (6) | 7 (3) |
| 4 | 375 | 300 | 5 | 40 (5) | 7 (3) |

TABLE 9
Percent (%) precisely edited reads at the Acetyl-CoA carboxylase target
locus (ACCase A2004V) in individual plantlets from independent
events scored as positive in the 2^{nd} edit PCR

| | NGS on individual shoots from | | |
|---|---|---|---|
| | independent events, positive in | | |
| | the 2nd edit PCR | | |
| | Target | % edit | % WT |
| Event name | reads | reads | reads |
| TMTA0166-0005-B01-06$001 | 55817 | 10.79 | 0.09 |
| TMTA0170-0097-B01-07$001 | 51820 | 16.32 | 51.08 |
| TMTA0170-0118-B01-09$001 | 54705 | 14.06 | 0.08 |
| TMTA0170-0119-B01-02$001 | 48846 | 18.39 | 0.15 |
| TMTA0166-0134-B01-02$001 | 52468 | 16.34 | 32.31 |
| TMTA0167-0135-B01-05$001 | 56139 | 14.72 | 13.36 |
| TMTA0167-0150-B01-01$001 | 53638 | 14.27 | 13.11 |
| TMTA0167-0152-B01-08$001 | 47913 | 40.21 | 0.04 |
| TMTA0167-0164-B01-05$001 | 44855 | 13.76 | 10.3 |
| TMTA0167-0163-B01-04$001 | 56177 | 15.62 | 39.62 |
| TMTA0167-0247-B01-03$001 | 53868 | 19.72 | 33.08 |
| TMTA0167-0235-B01-01$001 | 48851 | 9.16 | 63.89 |
| TMTA0167-0100-B01-04$001 | 59993 | 12.71 | 48.52 |
| TMTA0167-0188-B01-02$001 | 53936 | 13.45 | 17.07 |
| TMTA0167-0124-B01-09$001 | 55733 | 2.97 | 67.63 |
| TMTA0167-0140-B01-02$001 | 51273 | 1.93 | 77.74 |
| TMTA0167-0102-B01-03$001 | 57154 | 24.86 | 31.89 |
| TMTA0167-0211-B01-02$001 | 51305 | 64.06 | 0.01 |
| TMTA0167-0191-B01-09$001 | 56996 | 22.33 | 26.19 |
| TMTA0167-0214-B01-08$001 | 42659 | 14.99 | 37.49 |
| TMTA0167-0213-B01-01$001 | 59588 | 10.25 | 23.7 |

### Example 5: Homology-Dependent Precise Gene Editing for the Introduction of the ALSW548L Mutation in the ALS (Acetolactate Synthase) Gene of Allohexaploid Wheat by a Cas9 Nuclease

By using a Cas9 nuclease and a pre-screened gRNA/donor DNA combination for its capability of potential HR-mediated precise gene editing capability in allohexaploid wheat as described in example 3, we recovered edited wheat plants having the desired amino acid substitution W548L in one or more alleles of the ALS gene by HR-mediated donor of a targeted DSB and via indirect selection for resistance to PPT. We identified 2 appropriate sgRNA vectors. The sgRNA vectors pBAY02533 (SEQ ID NO: 11) and pBAY02535 (SEQ ID NO: 12) comprise a cassette for expression of the gRNA that guides the Cas9 nuclease for the creation of a DSB at the target site TS sequence GAACAACCAGCATCTGGGAA-TGG (SEQ ID NO: 56) and ATCTGGGAATGGTGGTGCAG-TGG (SEQ ID NO: 57), respectively. The donor DNA pBAY02542 (SEQ ID NO: 17) was designed for the introduction of 2 base substitutions at the target codon (TGG to CTC) leading to the W548L change at the protein level. The donor DNA includes an 805 bp DNA fragment of Triticum aestivum, cv. Fielder subgenome D, ALSgene containing the desired mutation (W548L substitution). The donor DNA contains also some other silent mutations to prevent cleavage of the donor DNA and the edited allele with the desired mutation (W548L). The 3-bp (CTC) core sequence in the donor DNA was flanked with an ˜400-bp left and right homologous arm, which are identical to the WT ALS sequence of the subgenome D.

Immature embryos, 2-3 mm size, were isolated from sterilized ears of wheat cv. Fielder and bombarded using the PDS-1000/He particle delivery system as described by Sparks and Jones (Cereal Genomics: Methods in Molecular biology, vol. 1099, Chapter 17). Plasmid DNA of vectors pBAY02430 (Cas9 nuclease) (SEQ ID NO: 1), pBAY02533 (gRNA) (SEQ ID NO: 11) or pBAY02535 (gRNA) (SEQ ID NO: 12), pBAY02542 (donor DNA) (SEQ ID NO: 17) were mixed with the plasmid pIB26 (SEQ ID NO: 18). The vector pIB26 (SEQ ID NO: 18) contains an egfp-bar fusion gene under control of the 35S promoter. Bombarded immature embryos were transferred to non-selective callus induction medium for 1-2 weeks, then moved to PPT containing selection media and PPT resistant calli were selected and transferred to regeneration media for shoot formation as described by Ishida et al. (Agrobacterium Protocols: Volume 1, Methods in Molecular Biology, vol. 1223, Chapter 15). All plants developed from one immature embryo were treated as a pool. Genomic DNA was extracted from pooled leaf samples and a primer pair (HT-18-135 Forward (SEQ ID NO: 32)/HT-18-136 Reverse (SEQ ID NO: 33)) was designed for specific amplification of the edited ALS gene. The plantlets in a pool that gave the expected PCR fragment in this 1^{st} edit specific PCR, were then transferred to individual tubes and further analyzed by PCR using primer pair HT-18-135 Forward (SEQ ID NO: 32)/HT-18-136 Reverse (SEQ ID NO: 33) and by deep sequencing. For 4 experiments a total of 325, 467, 385 and 339 embryos were bombarded with a mixture of plasmid DNA of pBAY02430 (Cas9 nuclease) (SEQ ID NO: 1), pBAY02533 (gRNA) (SEQ ID NO: 11) or pBAY02535 (SEQ ID NO: 12) and pBAY02542 (donor DNA) (SEQ ID NO: 17) and pIB26 (SEQ ID NO: 18). In these 4 experiments, phosphinotricin (PPT) tolerant shoot regenerating calli were obtained from in total 235, ˜258, 112 and 164 immature embryos, respectively. Specific amplification of the edited ALS gene was observed in 10, 11, 3 and 4 pooled leaf samples. A 2^{nd} edit specific PCR was performed on in total 53, 71, 27 and 13 individual plants derived from 10, 11, 3 and 3 plantlet pools scored as positive in the 1^{st} edit PCR and specific amplification of the edited ALS gene was observed in 14, 25, 12 and 4 individual plantlets, derived from 4, 7, 3 and 2 plantlet pools, respectively (Table 10). On a number of plants from independent events scored as positive in the 2^{nd} edit PCR, deep sequencing was performed. For the 1^{st} PCR primer pair HT-18-130 (SEQ ID NO: 31)/HT-18-136 (SEQ ID NO: 33) was used; these primers were positioned outside the homology arms of the donor DNA for the amplification of a 1278 bp fragment. For the nested PCR to amplify a region of 320 bp for NGS, primer pair HT-18-065 (SEQ ID NO: 23)/HT-18-066 (SEQ ID NO: 24) was used. These data showed that we have recovered plants with one or two alleles precisely edited with the desired AA substitution W548L. Plantlets with a precise edit % below 10% are considered as chimeric ones (e.g. TMTA0158-0107-B01-01$001, TMTA0183-0055-B01-01$001) (Table 11).

TABLE 10
Number of ALS W548L edited plantlets based on edit PCR analysis

| | | | # | # plantlets | if individual |
|---|---|---|---|---|---|
| | | PPTR | positive | tested in 2nd | plantlets |
| | | shoot | leaf | edit PCR, | positive in |
| | # bom- | re- | pools in | ide rived | 2nd edit PCR, |
| | barded | generating | 1st edit | from # leaf | (derived from # |
| Exp n° | embryos | calli | PCR | pools^{*}) | of leaf pools') |
| 1 | 325 | ~285 | 10 | 53 (10) | 14 (4) |
| 2 | 467 | ~308 | 11 | 71 (11) | 35 (7) |
| 3 | 385 | 112 | 3 | 27 (3} | 12 (3) |
| 4 | 333 | 164 | 4 | 13 (3) | 4 (2) |

TABLE 11
Percent (%) precisely edited reads at the Acetolactate synthase
gene (ALS W548L) in individual plantlets from independent events
scored as positive in the 2^{nd} edit PCR

| | NGS on individual shoots from inde- | | |
|---|---|---|---|
| | pendant events, positive in the 2nd | | |
| | edit PCR | | |
| | | % edit | % WT |
| Event name | Target reads | reads | reads |
| TMTA0158-0107-B01-01$001 | 50207 | 3.95 | 60.56 |
| TMTA0180-0050-B01-06$001 | 53374 | 21.69 | 0 |
| TMTA0176-0033-B01-04$001 | 57042 | 21.09 | 0 |
| TMTA0176-0032-B01-01$001 | 52353 | 21.71 | 0 |
| TMTA0176-0031-B01-01$001 | 43073 | 21.7 | 0 |
| TMTA0176-0225-B01-01$001 | 49785 | 22.72 | 0.01 |
| TMTA0176-0279-B01-01$001 | 47708 | 11.02 | 0 |
| TMTA0183-0055-B01-01$001 | 23655 | 5.86 | 0 |

### Example 6: Homology-Dependent Precise Gene Editing for the Introduction of the I1781L Mutation in the ACCase (Acetyl-CoA Carboxylase) Gene of Allohexaploid Wheat by a Cas9 Nuclease and by Direct Selection

Bombarded immature embryos were bombarded with a mixture of the plasmid DNAs pBAY02430 (Cas9) (SEQ ID NO: 1), pBAY02528 (gRNA) (SEQ ID NO: 5) and donor DNA pBAY02539 (SEQ ID NO: 13) for the introduction of the I1781L mutation in the ACCase gene. Bombarded immature embryos were transferred to non-selective callus induction medium for 1-2 weeks, then moved to selection media with 200 and 300 nM quizalofop. Quizalofop tolerant lines have been recovered that were positive in the edit specific PCR using primer pair HT-18-111 Forward (SEQ ID NO: 28)/HT-18-112 Reverse (SEQ ID NO: 29). On a number of plants from independent events scored as positive in the 2nd edit PCR, deep sequencing was performed. These NGS data further confirms that these plants contain one or more precisely edited alleles with the desired AA substitution I1781L.

### Example 7: Homology-Dependent Precise Gene Editing for the Introduction of the I1781L Mutation in the ACCase (Acetyl-CoA Carboxylase) Gene of Allohexaploid Wheat by RNP-Mediated Delivery of CRISPR/Cas9 Components

To generate CRISPR/Cas9 RNP complexes the Cas9 protein (Alt-R® S.p. Cas9 Nuclease V3, IDT) and the sgRNA (Alt-R® CRISPR-Cas9 crRNA XT and Alt-R® CRISPR-Cas9 tracrRNA, IDT) were premixed according to the protocol of IDT (www.idtdna.com). The sgRNA was designed to target the sequence CTAGGTGTGGAGAACATACA-TGG (SEQ ID NO: 50) which is positioned over the target codon in ACCase.

Immature embryos, 2-3 mm size, were bombarded with a mixture of RNP and donor DNA pBay02539 (SEQ ID NO: 13) using the PDS-1000/He particle delivery system as described by Svitashev et al. 2016. Bombarded immature embryos were transferred to non-selective callus induction medium for 2 weeks, then moved to selection medium with 200 nM quizalofop. For 2 experiments a total of 298 and 302 embryos were bombarded with a mixture of RNP and donor DNA pBAY02539 (SEQ ID NO: 13). From these 2 experiments quizalofop tolerant lines were obtained from 16 and 9 immature embryos and specific amplification of the edited ACCase gene using primer pair HT-18-111 Forward (SEQ ID NO: 28)/HT-18-112 Reverse (SEQ ID NO: 29) was observed for these 25 lines.

For 9 independent events scored as positive in the edit PCR, deep sequencing was performed on 1 plant/event. The region surrounding the intended target site was PCR amplified with Q5 High-Fidelity polymerase (M0492L) by means of nested PCR. For the 1st PCR primer pair HT-18-162 (SEQ ID NO: 34)/HT-18-112 (SEQ ID NO: 29) was used; these primers were positioned outside the homology arms of the donor DNA for the amplification of a 1736 bp fragment. For the nested PCR to amplify a region of a 386 bp for NGS, primer pair HT-18-048 (SEQ ID NO: 19)/HT-18-053 (SEQ ID NO: 21) was used. We assessed editing frequency by calculating the percentage of sequence reads showing evidence for presence of the desired mutations AA substitution (ACCase 11781 L) at the target codon as directed by the donor DNA, as a proportion of the total number of reads. These data showed that we have recovered plants with one to three alleles precisely edited with the desired AA substitution I1781L (Table 12).

TABLE 12
Percent (%) precisely edited reads at the at the Acetyl-CoA carboxylase
target locus (ACCase I1781L) in individual plantlets from
independent events scored as positive in the 2nd edit PCR

| | NGS on individual shoots | | |
|---|---|---|---|
| | from independant events, | | |
| | positive in the 2nd edit PCR | | |
| | Target | % | % |
| Event name | reads | edit reads | WT reads |
| TMTA0406-0002-B01-05$001 | 32333 | 20.15 | 71.48 |
| TMTA0406-0005-B01-02$001 | 24434 | 41.73 | 0 |
| TMTA0407-0002-B01-02$001 | 34153 | 35.65 | 18.29 |
| TMTA0407-0004-B01-06$001 | 29263 | 20.05 | 16.86 |
| TMTA0407-0008-B01-06$001 | 30420 | 18.72 | 29.71 |
| TMTA0407-0015-B01-07$001 | 23696 | 34.95 | 37.07 |
| TMTA0407-0018-B01-03$001 | 24723 | 23.44 | 0 |
| TMTA0407-0026-B01-01$001 | 28637 | 18.92 | 29.05 |
| TMTA0407-0027-B01-02$001 | 29306 | 20.59 | 60.67 |

### Example 8: Homology-Dependent Precise Gene Editing for the Introduction of the I1781L Mutation in the ACCase (Acetyl-CoA Carboxylase) Gene of Allohexaploid Wheat by a Paired Cas9 Nickase by RNP-Mediated Delivery of CRISPR/Cas9 Components

To generate CRISPR/Cas9 nickase RNP complexes the Cas9 nickase protein (Alt-R® S.p. Cas9 D10A Nickase V3, IDT) and each sgRNA (Alt-R® CRISPR-Cas9 crRNA XT and Alt-R® CRISPR-Cas9 tracrRNA, IDT) were premixed according to the protocol of IDT (www.idtdna.com). The crRNA1 was designed to target the sequence CTAGGTGTGGAGAACATACA-TGG (TS1) (SEQ ID NO: 50) and the crRNA2 was designed to target the target sequence CTCCTCATAGGCCCTAGAAT-AGG (TS2) (SEQ ID NO: 53) which are positioned on opposite strands with a distance of 32 nt between the 2 nick sites. Immature embryos, 2-3 mm size, were bombarded with a 1:1 mixture of RNP1 targeting TS1 and RNP2 targeting TS2 together with the donor DNA pBay02540 (SEQ ID NO: 14) using the PDS-1000/He particle delivery system as described by Svitashev et al. 2016. Bombarded immature embryos were transferred to non-selective callus induction medium for 2 weeks, then moved to selection medium with 200 nM quizalofop. Quizalofop resistant plants were further analyzed by PCR using primer set (HT-18-112/HT-18-113) (SEQ ID NOs: 29; 30) for specific amplification of the edited ACCase gene. On plants scored as positive in the edit PCR, deep sequencing was performed. For the deep sequencing the region surrounding the intended target site was PCR amplified with Q5 High-Fidelity polymerase (M0492L) by means of nested PCR. For the 1st PCR primer pair HT-18-162/HT18-112 (SEQ ID NOs: 34; 29) was used; these primers were positioned outside the homology arms of the donor DNA. For the nested PCR, primer pair HT-18-048/HT-18-053 (SEQ ID NOs: 19; 21) was used.

These data show that in nearly all plants containing allele(s) with the desired edit (I1781L), no alleles with NHEJ-derived InDels were present (Table 13).

TABLE 13
Percent (%) precisely edited reads at the at the Acetyl-CoA carboxylase
target locus (ACCase I1781L) in quizalofop resistant plants
edited by a paired Cas9 nickase delivered as RNP

| | | | | no | |
|---|---|---|---|---|---|
| | NGS | | | alleles | # |
| | Target | % I1781L | % | with | InDel |
| Plant name | reads | edits | WT | InDels | alleles |
| TMTA0496-0002-B01- | 18598 | 17.47 | 77.87 | X | 0 |
| 05$001 | | | | | |
| TMTA0496-0002-B01- | 20550 | 17.24 | 78.09 | X | 0 |
| 06$001 | | | | | |
| TMTA0497-0049-B01- | 21083 | 21.04 | 74.15 | X | 0 |
| 01$001 | | | | | |
| TMTA0497-0164-B01- | 24065 | 16.76 | 78.35 | X | 0 |
| 02$001 | | | | | |
| TMTA0497-0164-B01- | 20158 | 17.04 | 77.98 | X | 0 |
| 05$001 | | | | | |
| TMTA0497-0164-B01- | 21306 | 10.96 | 83.58 | X | 0 |
| 14$001 | | | | | |
| TMTA0497-0164-B01- | 25632 | 16.97 | 78.4 | X | 0 |
| 16$001 | | | | | |
| TMTA0498-0001-B01- | 23001 | 14.84 | 80.44 | X | 0 |
| 01$001 | | | | | |
| TMTA0498-0001-B01- | 21526 | 16.97 | 78.2 | X | 0 |
| 02$001 | | | | | |
| TMTA0543-0010-B01-02 | 121507 | 14.24 | 45.75 | | 1 |

### Example 9: Homology-Dependent Precise Gene Editing for the Introduction of the I1781L Mutation in the ACCase (Acetyl-CoA Carboxylase) Gene of Allohexaploid Wheat by a Paired Cas9 Nickase with Greater Distances Between the Nicks

For this experiment gRNAs are designed leading the SpCas9 nickase to target sites on opposite strands with the distance between the 2 nick sites of either 45 nt or 136 nt. Immature embryos were co-bombarded with the Cas9 nickase vector pBas02734 (SEQ ID NO: 3), the donor DNA pBas04096 (SEQ ID NO: 35) and the gRNA vector pair pBay02528 (SEQ ID NO: 5) and pBas04093 (SEQ ID NO: 37) for the creation of a nick on opposite strands at a distance of 136 nt from each other, or the embryos were co-bombarded with the Cas9 nickase vector pBas02734 (SEQ ID NO: 3), the donor DNA pBay02544 (SEQ ID NO: 36) and the gRNA vector pair pBay02529 (SEQ ID NO: 6) and pBay02531 (SEQ ID NO: 8) each creating a nick on opposite strands at a distance of 45 nt from each other. After bombardment immature embryos were transferred to non-selective callus induction medium for 2 weeks, then moved to selection medium with 200 nM quizalofop. Quizalofop resistant plants were further analyzed by PCR using primer set (HT-18-113 Forward/HT-18-112 Reverse) (SEQ ID NOs: 30; 29) for specific amplification of the edited ACCase gene. On plants scored as positive in the edit PCR, deep sequencing was performed. For the deep sequencing the region surrounding the intended target site was PCR amplified with Q5 High-Fidelity polymerase (M0492L) by means of nested PCR. For the 1st PCR primer pair HT18-162/HT-18-112 (SEQ ID NO: 34; 29) was used; these primers were positioned outside the homology arms of the donor DNA for the amplification of a 1736 bp fragment. For the nested PCR, primer pair 18-048/HT-18-053 (SEQ ID NOs: 19; 21) was used. These data in Table 14 showed that it is possible, even with larger distances between the nicks, to identify plants with one precisely edited allele carrying no alleles with NHEJ-derived InDels.

TABLE 14
Percent (%) precisely edited reads at the at the Acetyl-CoA
carboxylase target locus (ACCase I1781L) in quizalofop
resistant plants edited by a paired Cas9 nickase

| | distance | | % | | # |
|---|---|---|---|---|---|
| | between | Target | I1781L | % | INDEL |
| Plant name | nicks | reads | edits | WT | alleles |
| TMTA0279-0117- | 45 nt | 75258 | 24.19 | 73.29 | 0 |
| B01-01 | | | | | |
| TMTA0279-0128- | 45 nt | 79808 | 13.22 | 37.31 | 3 |
| B01-01 | | | | | |
| TMTA0280-0153- | 45 nt | 76765 | 19.71 | 78.05 | 0 |
| B01-01 | | | | | |
| TMTA0654-0022- | 136 nt | 122904 | 16.59 | 77.96 | 0 |
| B01-02 | | | | | |
| TMTA0654-0022- | 136 nt | 112145 | 18.06 | 75.84 | 0 |
| B01-03 | | | | | |

### Example 10: Homology-Dependent Precise Gene Editing for the Introduction of the TIPS Mutation in the 5-Enolpyruvylshikimate-3-Phosphate Synthase Gene in Rice

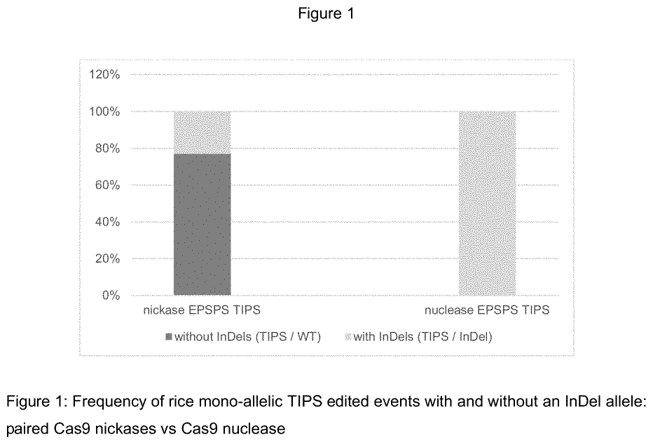

The following example describes homology-dependent precise gene editing by a paired nickase for the introduction of the T1731 and P177S mutation in the 5-enolpyruvylshikimate-3-phosphate synthase gene of Oryza sativa, providing the TIPS amino acid substitutions, conferring resistance to glyphosate. By using a rice codon optimized version of the Cas9 nickase (D10A) (pKVA824 (SEQ ID NO: 43)) and 2 gRNAs (pKVA766 (SEQ ID NO: 45)) and pKVA769 (SEQ ID NO: 46)) and a donor DNA (pKVA791 (SEQ ID NO: 47)), the desired mutations could be introduced in the target codons. The two sgRNAs were designed for the generation of 33 bp 3′ overhangs spanning the target codon. The sgRNA vectors pKVA766 and pKVA769 lead the SpCas9 nickase to the target sites TS1 (5′-CCATTGACAGCAGCCGTGACTGC-3′) (SEQ ID NO: 58) and TS2 (5′-GAGGAAGTGCAACTCTTCTTG-GGG 3′) (SEQ ID NO: 59), respectively. The sequence of exon 2 in the donor plasmid pKVA791 contained the TIPS amino acid nucleotide substitutions C518T, and C529T, and a silent mutation A531G to create a unique Pvul restriction site. Rice embryogenic callus derived from mature seeds was used as starting material for particle bombardment. Embryogenic callus was bombarded using the particle inflow gun (PIG) system (Grayel). The bombardment parameters were as follows: diameter gold particles, 0.6 μm; target distance 17 cm, bombardment pressure 500 kPa, and for each plasmid DNA (Cas9, gRNA, donor DNA) 1.25 μg DNA was used per shot. After bombardment the callus pieces were transferred to non-selective RSK500 callus induction medium (SK-1m salts Duchefa (Khanna & Raina, 1998, Plant Cell, Tissue and Organ Culture, 52: 145-153), Khanna vitamins (Khanna & Raina, supra), L-proline 1.16 g/L, CuSO4.5H2O 2.5 mg/L, 2.4-D 2 mg/L, maltose 20 g/L, sorbitol 30 g/L, MES 0.5 g/L, agarose 6 g/L, pH 5.8) for a few days, followed by transfer to RSK500 medium supplemented with 150 mg/L glyphosate. Shoots were regenerated from the active growing glyphosate tolerant embryogenic callus lines. Restriction digestion (Pvul) of the amplified PCR product over the target region of glyphosate tolerant events was done as a first molecular screen to confirm the introduction of the TIPS mutation in the native epsps gene. A silent mutation to create a Pvul site was introduced close to the TIPS mutation in the donor DNA to facilitate molecular screening for identification of TIPS edited events. Pvul digest of the amplified PCR product of 24 glyT events reveal 13 mono-allelic TIPS edited events, 10 bi-allelic TIPS edited events and 1 event with no TIPS mutation. Sequencing analysis of the bi-allelic events confirmed the presence of the TIPS mutation in both alleles. Sequencing of cloned PCR products obtained from 13 mono-allelic edited events obtained by the paired nickase showed that 10 of these events were mono-allelic TIPS edited events with one allele precisely edited with the TIPS mutation and one WT allele (TIPS/WT). The other 3 events had also a precisely edited TIPS allele but a non-specific mutation (InDel) in the other allele (TIPS/InDel) (FIG. 1).

Sequencing of cloned PCR products obtained from 23 mono-allelic TIPS edited events obtained by co-delivery of the Cas9 nuclease (pKVA790 (SEQ ID NO: 48)), the single sgRNA (pKVA766 (SEQ ID NO:45)) and the repair DNA (pKVA761 (SEQ ID NO: 60) instead of the paired Cas9 nickase as described above, showed that all these 23 events with one allele precisely edited with the TIPS mutation, also contained an InDel allele (TIPS/InDel) (FIG. 1). These data showed that by using a paired nickase instead of a nuclease, the number of (TIPS/WT) events is increased and the number of (TIPS/InDel) events reduced.

1. A method for introducing at least one donor DNA molecule into at least one target region of the genome of a plant cell comprising the steps of
a. introducing into said plant cell
i. a donorDNA molecule and
ii. at least one RNA guided nickase and
iii. at least two sgRNAs or at least two crRNA and tracrRNA and
b. incubating the plant cell to allow for introduction of said at least one donor DNA into said at least one target region of the genome, and
c. selecting a plant cell comprising the sequence of the donor DNA molecule in said target region,
wherein the nickases creates at least two nicks on opposite strands at the target site of the genomic DNA of the plant cell and
wherein these nicks are at least 20 apart from each other.

2. A method for producing a plant comprising a donor DNA comprising the steps of
a. introducing into a cell of said plant
i. a donorDNA molecule and
ii. at least one RNA guided nickase and
iii. at least two sgRNAs or at least two crRNA and tracrRNA and
b. incubating the plant cell to allow for introducing said at least one donor DNA into the target region of the genome of said plant cell, and
c. selecting a plant cell comprising the sequence of the donor DNA molecule in said target region, and
d. regenerating a plant from said selected plant cell,
wherein the nickase creates at least two nicks on opposite strands at the target site of the genomic DNA of the plant cell and
wherein these nicks are at least 20 bases apart from each other.

3. The method of claim 1, wherein after step b. the plant cell is incubated on a medium comprising a selection agent.

4. The method of claim 1, wherein the RNA guided nickase is a Cas nickase.

5. The method of claim 1, wherein the Cas nickase is a Cas9 or Cas12a nickase.

6. The method of claim 1, wherein at least one of the nickase or the sgRNA or crRNA and tracrRNA is introduced into said cell encoded by a nucleic acid molecule.

7. The method of claim 6, wherein the nucleic acid molecule is a plasmid comprising an expression cassette encoding said nickase or the sgRNA or crRNA and tracrRNA.

8. The method of claim 6, wherein the nucleic acid is an RNA molecule.

9. The method of claim 6, t 8 wherein the at least one nickase is sequence optimized for expression in the respective plant cell.

10. The method of claim 1, wherein at least one of the RNA guided nickase or the sgRNA or crRNA and tracrRNA are introduced into said cell as ribonucleoprotein (RNP) assembled outside said cell.

11. The method of claim 1, wherein a combination of donorDNA and crRNA/tracrRNA or sgRNA is preselected.

12. The method of claim 1, wherein the donor DNA and the RNA guided nickase and the single guideRNA or tracrRNA and crRNA are introduced into said cell using particle bombardment or Agrobacterium mediated introduction of DNA.

13. The method of claim 1, wherein the RNA guided nickase is comprising a nuclear localization signal.

14. The method of claim 2, wherein after step b. the plant cell is incubated on a medium comprising a selection agent.

15. The method of claim 2, wherein the RNA guided nickase is a Cas nickase.

16. The method of claim 2, wherein the Cas nickase is a Cas9 or Cas12a nickase.

17. The method of claim 2, wherein at least one of the nickase or the sgRNA or crRNA and tracrRNA is introduced into said cell encoded by a nucleic acid molecule.

18. The method of claim 17, wherein the nucleic acid molecule is a plasmid comprising an expression cassette encoding said nickase or the sgRNA or crRNA and tracrRNA.

19. The method of claim 17, wherein the nucleic acid is an RNA molecule.

20. The method of claim 17, wherein the at least one nickase is sequence optimized for expression in the respective plant cell.

21. The method of claim 2, wherein at least one of the RNA guided nickase or the sgRNA or crRNA and tracrRNA are introduced into said cell as ribonucleoprotein (RNP) assembled outside said cell.

22. The method of claim 2, wherein a combination of donorDNA and crRNA/tracrRNA or sgRNA is preselected.

23. The method of claim 2, wherein the donor DNA and the RNA guided nickase and the single guideRNA or tracrRNA and crRNA are introduced into said cell using particle bombardment or Agrobacterium mediated introduction of DNA.

24. The method of claim 2, wherein the RNA guided nickase is comprising a nuclear localization signal.
